(19) United States
(12) Patent Application Publication
(10) Pub. No.: US 2019/0328624 A1
(43) Pub. Date: Oct. 31, 2019

(54) METHODS OF REMOVING SPORES

(71) Applicant: 3M INNOVATIVE PROPERTIES COMPANY, SAINT PAUL, MN (US)

(72) Inventors: RANJANI V. PARTHASARATHY, WOODBURY, MN (US); CATHERINE D. HEAPY, NORTH ST. PAUL, MN (US)

(73) Assignee: 3M INNOVATIVE PROPERTIES COMPANY, SAINT PAUL, MN (US)

(21) Appl. No.: 16/475,283

(22) PCT Filed: Jan. 2, 2018
(86) PCT No.: PCT/US2018/012034

§ 371 Date: Jul. 1, 2019

Related U.S. Application Data
(60) Provisional application No. 62/442,044, filed on Jan. 4, 2017.

(51) Int. Cl. A61K 8/02; A61L 2/18; A61L 2/00; A61L 2/22; A61Q 17/00; A61L 2/232; A61K 8/34; A61K 8/84

(52) U.S. Cl. CPC A61K 8/0208; A61L 2/18; A61L 2/0088; A61L 2/22; A61K 2800/884; A61L 2/232; A61K 8/34; A61K 8/84; A61Q 17/005

(57) ABSTRACT

Disclosed herein are methods that include contacting a skin surface with a first liquid composition; and then contacting the skin surface with a cationic coated article loaded with a second liquid composition, while at least some portion of the first liquid composition remains on the skin surface, wherein at least one and only one of the first or the second composition comprises greater than or equal to 60 wt % of at least one alcohol.

### FIELD

The present disclosure relates to methods of removing spores from surfaces.

### BACKGROUND

There is a great deal of interest and urgency in preventing the spread of C. difficile, particularly in medical settings such as hospitals. Patients in a hospital setting often times develop C. difficile infections during or shortly after a course of antibiotics. While it is relatively easy to kill the vegetative form of C. difficile, the spore form of C. difficile, can be very difficult to kill. New technologies are therefore needed to address the problem of preventing the spread of C. difficile, between patients, health care workers, and the environment.

### SUMMARY

Disclosed herein are methods that include contacting a skin surface with a first liquid composition; and then contacting the skin surface with a cationic coated article loaded with a second liquid composition, while at least some portion of the first liquid composition remains on the skin surface, wherein at least one and only one of the first or the second composition comprises greater than or equal to 60 wt % of at least one alcohol.

The above summary of the present disclosure is not intended to describe each disclosed embodiment or every implementation of the present disclosure. The description that follows more particularly exemplifies illustrative embodiments. In several places throughout the application, guidance is provided through lists of examples; examples can be used in various combinations. In each instance, the recited list serves only as a representative group and should not be interpreted as an exclusive list.

### BRIEF DESCRIPTION OF THE DRAWINGS

FIGS. 1A and 1B are schematic views of a mechanical wiping device used in testing wipes.

### DETAILED DESCRIPTION

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, “top” and “bottom” (or other terms like “upper” and “lower”) are utilized strictly for relative descriptions and do not imply any overall orientation of the article in which the described element is located.

As used in this specification and the appended claims, the singular forms “a”, “an”, and “the” encompass embodiments having plural referents, unless the content clearly dictates otherwise.

As used in this specification and the appended claims, the term “or” is generally employed in its sense including “and/or” unless the content clearly dictates otherwise. The term “and/or” means one or all of the listed elements or a combination of any two or more of the listed elements.

As used herein, “have”, “having”, “include”, “including”, “comprise”, “comprising” or the like are used in their open ended sense, and generally mean “including, but not limited to”. It will be understood that “consisting essentially of”, “consisting of”, and the like are subsumed in “comprising” and the like. For example, a conductive trace that “comprises” silver may be a conductive trace that “consists of” silver or that “consists essentially of” silver.

As used herein, “consisting essentially of,” as it relates to a composition, apparatus, system, method or the like, means that the components of the composition, apparatus, system, method or the like are limited to the enumerated components and any other components that do not materially affect the basic and novel characteristic(s) of the composition, apparatus, system, method or the like.

The words “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure, including the claims.

Also herein, the recitations of numerical ranges by endpoints include all numbers subsumed within that range (e.g., 1 to 5 includes 1, 1.5, 2, 2.75, 3, 3.80, 4, 5, etc. or 10 or less includes 10, 9.4, 7.6, 5, 4.3, 2.9, 1.62, 0.3, etc.). Where a range of values is “up to” a particular value, that value is included within the range.

Use of “first,” “second,” etc. in the description above and the claims that follow is not intended to necessarily indicate that the enumerated number of objects is present. For example, a “second” substrate is merely intended to differentiate from another substrate (such as a “first” substrate). Use of “first,” “second,” etc. in the description above and the claims that follow is also not necessarily intended to indicate that one comes earlier in time than the other.

As used herein the term “spore” refers to microbial spores.

As used herein, “polymer” is inclusive of a homopolymer, copolymer, terpolymer, and the like.

As used herein, the term “bound” or “binding” in reference to the cationic coating (e.g., the guanidinyl-containing polymer in the cationic coating) being bound to the substrate or binding the cationic coating to the substrate means that the cationic coating cannot be removed without destroying the substrate. The cationic coating can be chemically attached to the substrate or can be crosslinked around the fibers of the substrate such that the coating cannot be removed by peeling, dissolving in water or an organic solvent.

As used herein, “alkylene” refers to a divalent radical of an alkane and includes straight-chained, branched, and cyclic alkyl groups and includes both unsubstituted and substituted alkyl groups. Unless otherwise indicated, the alkyl groups typically contain from 1 to 20 carbon atoms. Examples of “alkyl” as used herein include, but are not limited to, methylene, ethylene, n-propylene, n-butylene, n-pentylene, isobutylene, t-butylene, isopropylene, n-octylene, n-heptylene, ethylhexylene, cyclopentylene, cyclohexylene, cycloheptylene, adamantylene, and norbornylene, and the like.

As used herein, “aryl” is a monovalent radical of an aromatic group containing 5-12 ring atoms and can contain optional fused rings, which may be saturated, unsaturated, or aromatic. Examples of an aryl groups that are carbocylic include phenyl, naphthyl, biphenyl, phenanthryl, and anthracyl. The term “heteroaryl” refers to an aryl containing 1-3 heteroatoms such as nitrogen, oxygen, or sulfur and can contain fused rings. Some examples of heteroaryl groups are pyridyl, furanyl, pyrrolyl, thienyl, thiazolyl, oxazolyl, imidazolyl, indolyl, benzofuranyl, and benzthiazolyl. The term “(hetero)aryl” refers to both aryl and heteroaryl groups.

As used herein, “arylene” is a divalent radical of an aromatic group containing 5-12 ring atoms and can contain optional fused rings, which may be saturated, unsaturated, or aromatic. Examples of an arylene groups that are carbocylic include phenylene, naphthylene, biphenylene, phenanthrylene, and anthracylene. The term “heteroarylene” refers to an arylene containing 1-3 heteroatoms such as nitrogen, oxygen, or sulfur and can contain fused rings. Some examples of heteroarylene groups are pyridylene, furanylene, pyrrolylene, thienylene, thiazolylene, oxazolylene, imidazolylene, indolylene, benzofuranylene, and benzthiazolylene. The term “(hetero)arylene” refers to both arylene and heteroarylene.

Spores can adhere strongly to surfaces such as skin and can be very difficult to remove. Methods for removing spores from surfaces are important because there is a great deal of interest and urgency in preventing the spread of C. difficile, particularly in medical settings such as hospitals. Patients in a hospital setting often times develop C. difficile infections during or shortly after a course of antibiotics. While it is relatively easy to kill the vegetative form of C. difficile, the spore form of C. difficile, can be very difficult to kill. New technologies are needed to address the problem of preventing the spread of C. difficile, between patients, health care workers, and the environment.

Previously utilized methods or compositions are numerous, and include bleach, alcohol foams and gels, for example. Bleach is a commonly used sporicide, and is effective and recommended by the Centers for Disease Control (CDC) for use in a hospital setting to disinfect environmental surfaces. However, bleach cannot be utilized by patients and health care workers on their skin. Currently, alcohol foams and gels are used by most healthcare workers. These solutions are however not effective at eradicating C. difficile spores. The CDC recommendation for healthcare workers and patients affected by C. difficile is normal hand washing with soap, water and paper towel. However, it is not always convenient to implement this solution for a health care worker due to the lack of nearby sink access and time. Further, this is an inconvenient solution for the caregiver to implement on a patient with limited mobility.

Dilute bleach or hydrogen peroxide and similar products have been used on hands and have demonstrated the ability to kill spores. While spore kill is important, it is not clear that these solutions are safe for repeated use. For example, a health care worker may wash their hands 30-50 times in a single day, and some up to 75 times a day or more as they wash in and out of patient rooms. Long term toxicity and destruction of host tissues would be a concern for these solutions.

It is therefore extremely important to develop compositions that are safe for repeated use on the skin; reduce spores to a level equivalent to that of the CDC recommended protocol (soap, water and paper towel); and have the ability to kill vegetative bacteria rapidly. Alcohol is used to kill vegetative cells rapidly, but does not kill spores. It would also be useful to have compositions that could be used in various formats including wipes, gels, sprays, etc. Compositions that could be utilized for various aspects of patient care such as hand sanitization, patient bathing, and pre-op care could also be greatly beneficial. Compositions that could be used for patient care as well as environmental cleaning; for example, fragile or expensive equipment/surfaces in a hospital room that may warrant the use of less aggressive tissue friendly chemicals could also be quite useful to the medical community.

Disclosed methods overcome spore adhesion to surfaces, such as the skin, allowing for the spores to be dispersed and transferred into an article, for example a woven, knitted, or nonwoven wipe surface. Disclosed methods include methods of dislodging spores from a surface, methods of removing spores from a surface, or combinations thereof.

### First and Second Compositions

Disclosed methods can include at least two steps. Disclosed methods generally include a first step of contacting a surface with a first composition and a second step of contacting the surface with a second composition. Both the first and the second compositions are liquids. The second composition is generally loaded in or on a cationic coated article. The second composition is generally contacted with the surface while at least some of the first composition remains on the surface.

In some embodiments, at least one and only one of the first or second compositions include greater than or equal to 60 wt % of at least one alcohol. As such, in some embodiments, the first composition includes greater than or equal to 60 wt % alcohol and the second composition includes less than 60 wt % alcohol or no alcohol; or in some embodiments, the second composition includes greater than or equal to 60 wt % alcohol and the first composition includes less than 60 wt % alcohol or no alcohol. In some embodiments, at least one and only one of the first or second compositions includes alcohol. As such, in some embodiments, the first composition includes alcohol and the second composition does not; or in some embodiments, the second composition includes alcohol and the first composition does not.

In some embodiments the first composition, the second composition, or both can include a component that more easily allows the composition to wet a wipe such as a cationic coated wipe, for example a surfactant. Particular illustrative surfactants can include, for example nonionic surfactants such as sorbitan fatty acid esters or more specifically TWEEN®. Additionally, surfactants such as those discussed below with respect to optional components can also be utilized in the second composition.

In some embodiments the first composition, the second composition, or both can include an optional thickener. In some embodiments the first composition, the second composition, or both can include not greater than 0.2 wt % thickener.

### Alcohol

Disclosed methods utilize two compositions, a first composition and a second composition. In some embodiments, at least one and only one of the first and the second compositions include any amount of at least one alcohol. The one of the first and second compositions that includes no alcohol can include water as a solvent. The one of the first and second compositions that includes alcohol can include water as a co-solvent. In some embodiments, the one of the first and second compositions that includes no alcohol includes water as the main component (based on weight) of the solvent. In some embodiments, at least one and only one of the first and the second compositions includes greater than or equal to 60 wt % of at least one alcohol. The one of the first and second compositions that includes less than 60 wt % of at least one alcohol or no alcohol can include water as a solvent. The one of the first and second compositions that includes greater than or equal to 60 wt % of at least one alcohol can include water as a co-solvent. In some embodiments, the one of the first and second compositions that includes less than 60 wt % alcohol or no alcohol can include water as the main component (based on weight) of the solvent.

Any kind of alkyl monofunctional alcohol can be utilized as the alcohol(s) in the first or the second composition. In some embodiments, a lower hydrocarbon chain alcohol such as a C₂-C₅ alcohol can be utilized. In some embodiments the alcohol is chosen from ethanol and isopropanol, and in some embodiments the alcohol includes ethanol. Ethanol may be useful because it may provide a broad spectrum and quick kill of microbes and has an odor acceptable to consumers such as doctors, nurses and clinicians. Propyl alcohol (1-propanol) may also be used. A blend of two or more lower alcohols may also be utilized. The lower alcohols may be denatured, such as for example, denatured ethanol including SDA-3C (commercially available from Eastman Chemical, Kingsport, Tenn.). Co-solvents may be further included in the composition with the lower alcohol. Considering likely applications contemplated, suitable co-solvents can include for example acetone, hydrocarbons such as isooctane, glycols, ketones, ethers, and short chain esters.

In some embodiments, the amount of alcohol in the compositions can also be considered. The amount of alcohol in the compositions may be relevant because the alcohol content can provide bacteria killing properties, as well as spore removal via disclosed methods.

In some embodiments, one and only one of the first and second compositions can have not less than 60 wt % alcohol, or in some embodiments not less than 70 wt % alcohol based on the total weight of the entire first or second composition respectively. In some embodiments, one and only one of first or second compositions can have not greater than 85 wt % alcohol, not greater than 95 wt % alcohol, or not greater than 98% alcohol based on the total weight of the entire first or second composition respectively. In some embodiments one and only one of first or second compositions can have from 60 wt % to 70 wt % alcohol in the entire first or second composition based on the total weight of the entire first or second composition.

Alcohol amounts of not greater than 30 wt % may assist or aid in decreasing drying times of surfaces, e.g., skin surfaces, upon which the composition containing the 30 wt % alcohol is dispensed. Further, this amount of alcohol may not adversely or may not sufficiently adversely affect spore removal in this step. Including an amount of alcohol advantageous to drying time can be especially beneficial in a method where the first step of the method includes a first composition having an alcohol amount of greater than or equal to 60 wt %.

The investigators surprisingly found that the use of alcohol, which facilitates bacterial kill, does not decrease the effectiveness of spore removal. This was surprising to the investigators because alcohol was thought to hinder spore removal.

### Cationic Coated Article

Disclosed methods include a second step of contacting the surface with an article loaded with a second composition. Disclosed articles generally include a substrate that includes a cationic coating. The cationic coated substrate of the article is loaded with the second composition. The substrate can be porous and can include sponge, nonwoven fabric, or woven fabric, for example. In some embodiments, the article can be a wipe that includes a cationic coated substrate onto or into which the second composition is loaded.

The substrate may be in any suitable form for example. Some suitable substrates are woven or non-woven fabrics that are in the form of a sheet. The sheet can have any desired size and shape. Other suitable substrates are sponges that can have any desired size or shape. The substrates are usually porous. Suitable substrates are typically flexible so that the wipe can easily conform and contact various surfaces such as those that are not flat.

The substrate may be formed from any suitable thermoplastic or thermoset material. The material may be an organic polymeric material. Suitable organic polymeric materials include, but are not limited to, poly(meth)acrylates, poly(meth)acrylamides, polyolefins, poly(isoprenes), poly(butadienes), fluorinated polymers, chlorinated polymers, polyamides, polyimides, polyethers, poly(ether sulfones), poly(sulfones), poly(vinyl acetates), copolymers of vinyl acetate, such as poly(ethylene)-co-poly(vinyl alcohol), poly(phosphazenes), poly(vinyl esters), poly(vinyl ethers), poly(vinyl alcohols), poly(carbonates), polyurethanes, and cellulosic materials.

Suitable polyolefins include, but are not limited to, poly(ethylene), poly(propylene), poly(1-butene), copolymers of ethylene and propylene, alpha olefin copolymers (such as copolymers of ethylene or propylene with 1-butene, 1-hexene, 1-octene, and 1-decene), poly(ethylene-co-1-butene) and poly(ethylene-co-1-butene-co-1-hexene).

Suitable fluorinated polymers include, but are not limited to, poly(vinyl fluoride), poly(vinylidene fluoride), copolymers of vinylidene fluoride (such as poly(vinylidene fluoride-co-hexafluoropropylene), and copolymers of chlorotrifluoroethylene (such as poly(ethylene-co-chlorotrifluoroethylene).

Suitable polyamides include, but are not limited to, poly(iminoadipoyliminohexamethylene), poly(iminoadipoyliminodecamethylene), and polycaprolactam. Suitable polyimides include, but are not limited to, poly(pyromellitimide).

Suitable poly(ether sulfones) include, but are not limited to, poly(diphenylether sulfone) and poly(diphenylsulfone-co-diphenylene oxide sulfone).

Suitable copolymers of vinyl acetate include, but are not limited to, poly(ethylene-co-vinyl acetate) and such copolymers in which at least some of the acetate groups have been hydrolyzed to afford various poly(vinyl alcohols).

Suitable cellulosic materials include cotton, rayon, and blends thereof.

In some embodiments, the substrate is formed from propylene polymers (e.g., homopolymer or copolymers). Polypropylene polymers, particularly polypropylene homopolymers, may be especially useful for some applications due to properties such as non-toxicity, inertness, low cost, and the ease with which it can be extruded, molded, and formed into articles. Polypropylene polymers can be formed, for example, into porous sheets of woven or nonwoven fibers.

Some substrates are nonwoven fabrics. As used herein, the term “nonwoven fabric” refers to a fabric or web that has a structure of individual fibers or filaments that are randomly and/or unidirectionally interlaid in a mat-like fashion. The individual fibers or threads are not interlaid in an identifiable pattern as in a knitted or woven fabric. Examples of suitable nonwoven fabrics include, but are not limited to, melt-blown fabrics, spun-bond fabrics, carded fabrics, wetlaid fabrics, and air-laid fabrics.

Spun-bonded fibers are typically small diameter fibers that are formed by extruding molten thermoplastic polymer as filaments from a plurality of fine, usually circular capillaries of a spinneret with the diameter of the extruded fibers being rapidly reduced. Melt-blown fibers are typically formed by extruding the molten thermoplastic material through a plurality of fine, usually circular, die capillaries as molten threads or filaments into a high velocity, usually heated gas (e.g., air) stream which attenuates the filaments of molten thermoplastic material to reduce their diameter. Thereafter, the melt-blown fibers are carried by the high velocity gas stream and are deposited on a collecting surface to from a fabric of randomly disbursed melt-blown fibers. Any of the non-woven fabrics may be made from a single type of fiber or two or more fibers that differ in the type of thermoplastic polymer and/or thickness.

Wet-laid fibers can be formed into sheets by forming a slurry that contains a) fibers and b) a suspending liquid such as water, a water-miscible organic solvent, or a mixture thereof. The slurry is placed in a mold or deposited in a layer. The suspending liquid is removed to form a sheet or mat. The sheet or mat is then dried. In some embodiments, a polymeric binder is included in the dispersion. In other embodiments, a polymeric binder can be applied after formation of a sheet or mat. The polymeric binder is often a latex polymer.

Further details on the manufacturing method of nonwoven fabrics may be found in Wente, Superfine Thermoplastic Fibers, 48 INDUS. ENG. CHEM. 1342(1956), or in Wente et al., Manufacture Of Superfine Organic Fibers, (Naval Research Laboratories Report No. 4364, 1954).

The substrate includes a cationic coating or is coated with a composition that is cationic in nature. In some embodiments, the coatings can include chitosan, or polymers such as polyethylenimine (PEI), or quaternized cellulose, silanes, or guar gums, guanidinyl coatings, or combinations thereof. In some embodiments, the coatings can include guanidinyl coatings.

In some embodiments, cationic coatings can include a guanidinyl-containing polymer that can be bound to the substrate. Although any guanidinyl-containing polymer can be used in the cationic coating, in some embodiments, a polymer of Formula (I) can be utilized.

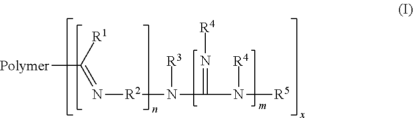

In Formula (I), the group R¹ is hydrogen, C₁-C₁₂ (hetero)alkyl, or C₅-C₁₂ (hetero)aryl, or a residue of the polymer chain. The group R² is a covalent bond, a C₂-C₁₂ (hetero)alkylene, or a C₅-C₁₂ (hetero)arylene. The group R³ is H, C₁-C₁₂ (hetero)alkyl, or C₅-C₁₂ (hetero)aryl, or can be a residue of the polymer chain when n is 0. Each group R⁴ is independently hydrogen, C₁-C₁₂ (hetero)alkyl, or C₅-C₁₂ (hetero)aryl. The group R⁵ is hydrogen, C₁-C₁₂ (hetero)alkyl, C₅-C₁₂ (hetero)aryl, or —N(R⁴)₂. The variable n is equal to 0 or 1 depending on the precursor polymer used to form the guanidinyl-containing polymer. The variable m is equal to 1 or 2 depending on whether the cationic group is a guanidinyl or biguanidinyl group. The term “Polymer” in Formula (I) refers to all portions of the guanidinyl-containing polymer except the x groups of formula —[C(R¹)═N—R²—]NN(R³)—[C(═NR⁴)—NR⁴R⁵-]ₘ. The term x is a variable equal to at least 1.

Most guanidinyl-containing polymers have more than one guanidinyl group. The number of guanidinyl groups can be varied depending the method used to prepare the guanidinyl-containing polymer. For example, the number of guanidinyl groups can depend on the choice of precursor polymer selected for reacting with a suitable guanylating agent. In some embodiments, the variable x can be up to 1000, up to 500, up to 100, up to 80, up to 60, up to 40, up to 20, or up to 10.

The guanidinyl-containing polymer of Formula (I) is often the reaction product of (a) a precursor polymer and (b) a suitable guanylating agent. The precursor polymer is often an amino-containing polymer or a carbonyl-containing polymer. When the precursor polymer is an amino-containing polymer, the variable n in Formula (I) is typically equal to 0. When the precursor polymer is a carbonyl-containing polymer, the variable n is equal to 1. If the guanylating agent contains a guanidinyl group or a precursor of a guanidinyl group, the variable m in Formula (I) is equal to 1. If the guanylating agent contains a biguanidinyl group or a precursor of a biguanidinyl group, the variable m in Formula (I) is equal to 2.

In embodiments where n is 0, the base polymer of the guanidinyl-containing polymer is often prepared by reaction of a suitable guanylating agent and an amino-containing polymer. In other embodiments, where n is 1, the guanidinyl-containing polymer is often prepared by reaction of a suitable guanylating agent and a carbonyl-containing polymer.

In those embodiments where n is 0 and the precursor polymer is an amino-containing polymer, the structure of the guanidinyl-containing polymer of Formula (I) can also be written more simply as the structure of Formula (II).

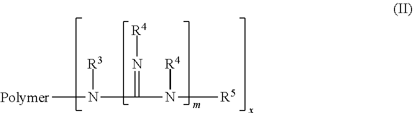

In Formula (II), the group R³ is hydrogen, C₁-C₁₂ (hetero)alkyl, or C₅-C₁₂ (hetero)aryl, or can be a residue of the polymer chain. When the guanidinyl group is part of a pendant group, R³ is hydrogen, C₁-C₁₂ (hetero)alkyl, or C₅-C₁₂ (hetero)aryl. Each R⁴ is independently hydrogen, C₁-C₁₂ (hetero)alkyl, or C₅-C₁₂ (hetero)aryl. The group R⁵ is hydrogen, C₁-C₁₂ (hetero)alkyl, or C₅-C₁₂ (hetero)aryl, or —N(R⁴)₂. The variable m is equal to 1 or 2. The term “Polymer” in Formula (II) refers to all portions of the guanidinyl-containing polymer except the x groups of formula —N(R³)—[C(═NR⁴)—NR⁴R⁵-]ₘ. The term x is a variable equal to at least 1.

The amino-containing polymer used as a precursor polymer to prepare a guanidinyl-containing polymer of Formula (II) can be represented by the formula Polymer —N(R³)H. As noted above, however, the amino-containing polymer typically has many groups —N(R³)H but Formula (I) shows only one for ease of discussion purposes only. The —N(R³)H groups can be a primary or secondary amino group and can be part of a pendant group or part of the backbone of the precursor polymer. The amino-containing polymers can be synthesized or can be naturally occurring biopolymers. Suitable amino-containing polymers can be prepared by chain growth or step growth polymerization procedures with amino-containing monomers. These monomers can also, if desired, be copolymerized with other monomers without an amino-containing group. Additionally, the amino-containing polymers can be obtained by grafting primary or secondary amine groups using an appropriate grafting technique.

In some embodiments, useful amino-containing polymers are polyamines that are water soluble or water-dispersible. As used herein, the term “water soluble” refers to a material that can be dissolved in water. The solubility is typically at least about 0.1 gram per milliliter of water. As used herein, the term “water dispersible” refers to a material that is not water soluble but that can be emulsified or suspended in water.

Examples of amino-containing polymers suitable for use, which are prepared by chain growth polymerization include, but are not limited to, polyvinylamine, poly(N-methylvinylamine), polyallylamine, polyallylmethylamine, polydiallylamine, poly(4-aminomethylstyrene), poly(4-aminostyrene), poly(acrylamide-co-methylaminopropylacrylamide), and poly(acrylamide-co-aminoethylmethacrylate).

Examples of amino polymers suitable for use, which are prepared by step growth polymerization include, but are not limited to, polyethylenimine, polypropylenimme, polylysine, polyaminoamides, polydimethylamine-epichlorohydrin-ethylenediamine, and any of a number of polyaminosiloxanes, which can be prepared from monomers such as aminopropyltriethoxysilane, N-(2-aminoethyl)-3-aminopropyltrimethoxysilane, N-trimethoxysilylpropyl-N-methylamine, and bis(trimethoxysilylpropyl)amine.

Other useful amino-containing polymers that have primary or secondary amino end groups include, but are not limited to, dendrimers (hyperbranched polymers) formed from polyamidoamine (PAMAM) and polypropylenimme. Exemplary dendrimeric materials formed from PAMAM are commercially available under the trade designation “STARBURST (PAMAM) dendrimer” (e.g., Generation 0 with 4 primary amino groups, Generation 1 with 8 primary amino groups, Generation 2 with 16 primary amino groups, Generation 3 with 32 primary amino groups, and Generation 4 with 64 primary amino groups) from Aldrich Chemical (Milwaukee, Wis.). Dendrimeric materials formed from polypropylenimme are commercially available under the trade designation “DAB-Am” from Aldrich Chemical. For example, DAB-Am-4 is a generation 1 polypropylenimme tetraamine dendrimer with 4 primary amino groups, DAB-Am-8 is a generation 2 polypropylenimme octaamine dendrimer with 8 primary amino groups, DAB-Am-16 is a generation 3 polypropylenimme hexadecaamine with 16 primary amino groups, DAB-Am-32 is a generation 4 polypropylenimme dotriacontaamine dendrimer with 32 primary amino groups, and DAB-Am-64 is a generation 5 polypropylenimme tetrahexacontaamine dendrimer with 64 primary amino groups.

Examples of suitable amino-containing polymers that are biopolymers include chitosan as well as starch that is grafted with reagents such as methylaminoethylchloride.

Still other examples of amino-containing polymers include polyacrylamide homo- or copolymers and amino-containing polyacrylate homo- or copolymers prepared with a monomer composition containing an amino-containing monomer such as an aminoalkyl(meth)acrylate, (meth)acrylamidoalkylamine, and diallylamine.

For some wipes, the preferred amino-containing polymers include polyaminoamides, polyethyleneimine, polyvinylamine, polyallylamine, and polydiallylamine.

Suitable commercially available amino-containing polymers include, but are not limited to, polyamidoamines that are available under the trade designations ANQUAMINE (e.g., ANQUAMINE 360, 401, 419, 456, and 701) from Air Products and Chemicals (Allentown, Pa.), polyethylenimine polymers that are available under the trade designation LUPASOL (e.g., LUPASOL FG, PR 8515, Waterfree, P, and PS) from BASF Corporation (Resselaer, N.Y.), polyethylenimine polymers such as those available under the trade designation CORCAT P-600 from EIT Company (Lake Wylie, S.C.), and polyamide resins such as those available from Cognis Corporation (Cincinnati, Ohio) under the traded designation VERSAMID series of resins that are formed by reacting a dimerized unsaturated fatty acid with alkylene polyamines.

Guanidinyl-containing polymers can be prepared by reaction of the amino-containing polymer precursor with a guanylating agent. Although all the amino groups of the amino-containing polymer can be reacted with the guanylating agent, there are often some unreacted amino groups from the amino-containing polymer precursor remaining in the guanidinyl-containing polymer. Typically, at least 0.1 mole percent, at least 0.5 mole percent, at least 1 mole percent, at least 2 mole percent, at least 10 mole percent, at least 20 mole percent, or at least 50 mole percent of the amino groups in the amino-containing polymer precursor are reacted with the guanylating agent. Up to 100 mole percent, up to 90 mole percent, up to 80 mole percent, or up to 60 mole percent of the amino groups can be reacted with the guanylating agent. For example, the guanylating agent can be used in amounts sufficient to functionalize 0.1 to 100 mole percent, 0.5 to 90 mole percent, 1 to 90 mole percent, 1 to 80 mole percent, 1 to 60 mole percent, 2 to 50 mole percent, 2 to 25 mole percent, or 2 to 10 mole percent of the amino groups in the amino-containing polymer.

Known guanylating agents for reaction with an amino-containing polymer precursor include, but are not limited to, cyanamide; O-alkylisourea salts such as O-methylisourea sulfate, O-methylisourea hydrogen sulfate, O-methylisourea acetate, O-ethylisourea hydrogen sulfate, and O-ethylisourea hydrochloride; chloroformamidine hydrochloride; 1-amidino-1,2,4-triazole hydrochloride; 3,5-dimethylpyrazole-1-carboxamidine nitrate; pyrazole-1-carboxamidine hydrochloride; N-amidinopyrazole-1-carboxamidine hydrochloride; and carbodiimides such as dicyclohexylcarbodiimide, N-ethyl-N′-(3-dimethylaminopropyl)carbodiimide, and diisopropylcarbodiimide. The amino-containing polymer may also be acylated with guanidino-functional carboxylic acids such as guanidinoacetic acid and 4-guanidinobutyric acid in the presence of activating agents such as EDC (N-[3-(dimethylamino)propyl]-3-ethylcarbodiimide hydrochloride), or EEDQ (2-ethoxy-1-ethoxycarbonyl-1,2-dihydroquinoline). Additionally, the guanidinyl-containing polymer may be prepared by alkylation with chloroacetone guanyl hydrazone, as described in U.S. Pat. No. 5,712,027 (Ali et al.).

Guanylating agents for the preparation of biguanide-containing polymers include sodium dicyanamide, dicyanodiamide and substituted cyanoguanidines such as N³-p-chlorophenyl-N¹-cyanoguanidine, N³-phenyl-N¹-cyanoguanidine, N³-alpha-naphthyl-N¹-cyanoguanidine, N³-methyl-N¹-cyanoguanidine, N³,N³-dimethyl-N¹-cyanoguanidine, N³-(2-hydroxyethyl)-N¹-cyanoguanidine, and N³-butyl-N¹-cyanoguanidine. Alkylene- and arylenebiscyanoguanidines may be utilized to prepare biguanide functional polymers by chain extension reactions. The preparation of cyanoguanidines and biscyanoguanidines is described in detail in Rose, F. L. and Swain, G. J. Chem Soc, 1956, pp. 4422-4425. Other useful guanylating reagents are described by Alan R. Katritzky et al., Comprehensive Organic Functional Group Transformation, Vol. 6, p. 640.

The guanidinyl-containing polymer formed by reaction of an amino-containing polymer precursor and a guanylating agent will have pendent or catenary guanidinyl groups of the Formula (III).

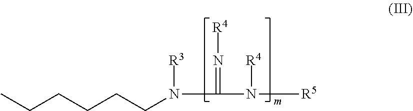

In Formula (III), the groups R³, R⁴, and R⁵ and the variable m are the same as defined above. The wavy line attached to the N(R³) group shows the position of attachment the group to the rest of the polymeric material. In most embodiments, the group of Formula (III) is in a pendant group of the guanidinyl-containing polymer.

In some embodiments, it may be advantageous to react the amino-containing polymer precursor to provide other ligands or groups in addition to the guanidinyl-containing group. For example, it may be useful to include a hydrophobic ligand, an ionic ligand, or a hydrogen bonding ligand. This can be particularly advantageous for the removal of certain microorganisms during the wiping of a microorganism-contaminated surface.

The additional ligands can be readily incorporated into the amino-containing polymers by alkylation or acylation procedures well known in the art. For example amino groups of the amino-containing polymer can be reacted using halide, sulfonate, and sulfate displacement reactions or using epoxide ring opening reactions. Useful alkylating agents for these reactions include, for example, dimethylsulfate, butyl bromide, butyl chloride, benzyl bromide, dodecyl bromide, 2-chloroethanol, bromoacetic acid, 2-chloroethyltrimethylammonium chloride, styrene oxide, glycidyl hexadecyl ether, glycidyltrimethylammonium chloride, and glycidyl phenyl ether. Useful acylating agents include, for example, acid chlorides and anhydrides such as benzoyl chloride, acetic anhydride, succinic anhydride, and decanoyl chloride, and isocyanates such as trimethylsilylisocyanate, phenyl isocyanate, butyl isocyanate, and butyl isothiocyanate. In such embodiments 0.1 to 20 mole percent, preferably 2 to 10 mole percent, of the available amino groups of the amino-containing polymer may be alkylated and/or acylated.

The guanidinyl-containing polymer can be crosslinked. The amino-containing polymer can be crosslinked prior to reaction with the guanylating agent. Alternatively, the guanidinyl-containing polymer can be crosslinked by reaction of a crosslinker with remaining amino groups from the amino-containing polymer precursor or with some of the guanidinyl groups. Suitable crosslinkers include amine-reactive compounds such as bis- and polyaldehydes such as glutaraldehyde, bis- and polygylcidylethers such as butanedioldiglycidylether and ethyleneglycoldiglycidylether, polycarboxylic acids and their derivatives (e.g., acid chlorides), polyisocyanates, formaldehyde-based crosslinkers such as hydroxymethyl and alkoxymethyl functional crosslinkers, such as those derived from urea or melamine, and amine-reactive silanes, such as 3-glycidoxypropyltrimethoxysilane, 3-glycidoxypropyltriethoxysilane, 5,6-epoxyhexyltriethoxysilane, (p-chloromethyl)phenyltrimethoxysilane, chloromethyltriethoxysilane, 3-isocyanatopropyltriethoxysilane, and 3-thiocyanatopropyltriethoxysilane.

In other embodiments, the guanidinyl-containing polymer is of Formula (IV), which corresponds to Formula (I) where n is equal to 1.

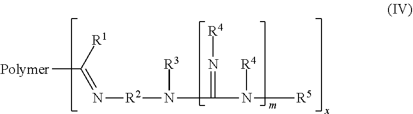

In Formula (IV), the group R¹ is hydrogen, C₁-C₁₂ (hetero)alkyl, or C₅-C₁₂ (hetero)aryl, or a residue of the polymer chain. If the guanidinyl-containing group is the reaction product of a guanylating agent and a carbonyl group that is part of the backbone of the polymer, R¹ is a residue of the polymer chain. Group R² is a covalent bond, a C₂-C₁₂ (hetero)alkylene, or a C₅-C₁₂ (hetero)arylene. Group R³ is hydrogen, C₁-C₁₂ (hetero)alkyl, or C₅-C₁₂ (hetero)aryl. Each R⁴ is independently H, C₁-C₁₂ (hetero)alkyl, or C₅-C₁₂ (hetero)aryl. Group R⁵ is hydrogen, C₁-C₁₂ (hetero)alkyl, or C₅-C₁₂ (hetero)aryl, or —N(R⁴)₂—. The variable m is equal to 1 or 2. The term “Polymer” in Formula (I) refers to all portions of the guanidinyl-containing polymer except the x groups of formula —C(R¹)═N—R²—N(R³)—[C(═NR⁴)—NR⁴R⁵-]ₘ. The term x is a variable equal to at least 1.

Guanidinyl-containing polymers of Formula (IV) are the reaction product of a carbonyl-containing polymer and a suitable guanylating agent for reaction with a carbonyl group. The carbonyl-containing polymer used as a precursor polymer to prepare a guanidinyl-containing polymer of Formula (IV) can be represented by the formula Polymer-C(O)—R¹ The carbonyl-containing polymer precursor typically has many groups —C(O)—R¹ but Formula (IV) shows only one for ease of discussion purposes only. The carbonyl group —C(O)—R¹ is an aldehyde group (when R¹ is hydrogen) or a ketone groups (when R¹ is a (hetero)alkyl or (hetero)aryl). Although the carbonyl-group can be part of the polymeric backbone or part of a pendant group from the polymeric backbone, it is typically in a pendant group.

In some embodiments, the carbonyl-containing polymer is the polymerized product of a monomer composition that includes an ethylenically unsaturated monomer having a carbonyl group, preferably a ketone group. Suitable monomers having a carbonyl group include, but are not limited to, acrolein, vinyl methyl ketone, vinyl ethyl ketone, vinyl isobutyl ketone, isopropenyl methyl ketone, vinyl phenyl ketone, diacetone (meth)acrylamide, acetonyl acrylate, and acetoacetoxyethyl (meth)acrylate.

In other embodiments, the carbonyl-containing polymer is the polymerized product of a monomer composition that includes carbon monoxide and one or more ethylenically unsaturated monomer (i.e., the carbonyl-containing polymer is a carbon monoxide copolymers). An example of a carbon monoxide containing copolymer is ELVALOY 741, a terpolymer of ethylene/vinyl acetate/carbon monoxide from DuPont (Wilmington, Del., USA).

In addition to carbon monoxide and/or an ethylenically unsaturated monomer with a carbonyl group (e.g., a ketone group), the monomer composition used to form that carbonyl-containing polymer can optionally further comprise ethylenically unsaturated hydrophilic monomer units. As used herein, “hydrophilic monomers” are those polymerizable monomers having water miscibility (water in monomer) of at least 1 weight percent preferably at least 5 weight percent without reaching a cloud point, and contain no functional groups that would interfere with the binding of biological substances to the ligand group. The carbonyl-containing polymer may include, for example, 0 to 90 weight percent of the hydrophilic monomers in the monomer composition. If present, the hydrophilic monomer can be present in an amount in a range of 1 to 90 weight percent, 1 to 75 weight percent, 1 to 50 weight percent, 1 to 25 weight percent, or 1 to 10 weight percent based on based a total weight of the monomer composition.

The hydrophilic groups of the hydrophilic monomers may be neutral, have a positive charge, a negative charge, or a combination thereof. Hydrophilic monomers with an ionic group can be neutral or charged depending on the pH conditions. Hydrophilic monomers are typically used to impart a desired hydrophilicity (i.e. water solubility or dispersibility) to the carbonyl-containing polymer. A negatively charged hydrophilic monomer may be included as long as it is in small enough amounts that it doesn't interfere with the binding interaction of the guanidinyl group.

Some exemplary hydrophilic monomers that are capable of providing a positive charge are amino (meth)acrylates or amino (meth)acrylamides of Formula (V) or quaternary ammonium salts thereof. The counter ions of the quaternary ammonium salts are often halides, sulfates, phosphates, nitrates, and the like.

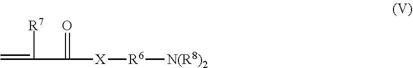

In Formula (V), the group X is oxy (i.e., -0-) or —NR³— where R³ is hydrogen, C₁-C₁₂ (hetero)alkyl, or C₅-C₁₂ (hetero)aryl. The group R⁶ is a C₂ to C₁₀ alkylene, preferably a C₂-C₆ alkylene. The group R⁷ is independently hydrogen or methyl. Each R⁸ is independently hydrogen, alkyl, hydroxyalkyl (i.e., an alkyl substituted with a hydroxy), or aminoalkyl (i.e., an alkyl substituted with an amino). Alternatively, the two R⁸ groups taken together with the nitrogen atom to which they are attached can form a heterocyclic group that is aromatic, partially unsaturated (i.e., unsaturated but not aromatic), or saturated, wherein the heterocyclic group can optionally be fused to a second ring that is aromatic (e.g., benzene), partially unsaturated (e.g., cyclohexene), or saturated (e.g., cyclohexane).

It will be understood with respect to Formula (V) that the depicted ethylenically unsaturated(meth)acryloyl group (CH₂═C(R⁷)—C(O)— group) may be replaced by another ethylenically unsaturated group of reduced reactivity, such as vinyl, vinyloxy, allyl, allyloxy, and acetylenyl.

In some embodiments of Formula (V), both R⁸ groups are hydrogen. In other embodiments, one R⁸ group is hydrogen and the other is an alkyl having 1 to 10, 1 to 6, or 1 to 4 carbon atoms. In still other embodiments, at least one of R⁸ groups is a hydroxy alkyl or an amino alkyl that have 1 to 10, 1 to 6, or 1 to 4 carbon atoms with the hydroxy or amino group being positioned on any of the carbon atoms of the alkyl group. In yet other embodiments, the R⁸ groups combine with the nitrogen atom to which they are attached to form a heterocyclic group. The heterocyclic group includes at least one nitrogen atom and can contain other heteroatoms such as oxygen or sulfur. Exemplary heterocyclic groups include, but are not limited to imidazolyl. The heterocyclic group can be fused to an additional ring such as a benzene, cyclohexene, or cyclohexane. Exemplary heterocyclic groups fused to an additional ring include, but are not limited to, benzoimidazolyl.

Illustrative amino acrylates (i.e., “X” in Formula (V) is oxy) include N,N-dialkylaminoalkyl (meth)acrylates such as, for example, N,N-dimethylaminoethyl(meth)acrylate, N,N-dimethylaminoethylacrylate, N,N-diethylaminoethylacrylate, N,N-dimethylaminopropyl(meth)acrylate, N-tert-butylaminopropyl(meth)acrylate, and the like.

Illustrative amino (meth)acrylamides (i.e., “X” in Formula (V) is —NR—) include, for example, N-(3-aminopropyl)methacrylamide, N-(3-aminopropyl)acrylamide, N-[3-(dimethylamino)propyl]methacrylamide, N-[3-(dimethylamino)propy 1]acrylamide, N-(3-imidazolylpropyl)methacrylamide, N-(3-imidazolylpropyl)acrylamide, N-(2-imidazolylethyl)methacrylamide, N-(1,1-dimethyl-3-imidazolylpropyl)methacrylamide, N-(1,1-dimethyl-3-imidazolylpropyl)acrylamide, N-(3-benzimidazolylpropyl)acrylamide, and N-(3-benzimidazolylpropyl)methacrylamide.

Illustrative quaternary salts of the monomers of Formula (V) include, but are not limited to, (meth)acrylamidoalkyltrimethylammonium salts (e.g., 3-methacrylamidopropyltrimethylammonium chloride and 3-acrylamidopropyltrimethylammonium chloride) and (meth)acryloxyalkyltrimethylammonium salts (e.g., 2-acryloxyethyltrimethylammonium chloride, 2-methacryloxyethyltrimethylammonium chloride, 3-methacryloxy-2-hydroxypropyltrimethylammonium chloride, 3-acryloxy-2-hydroxypropyltrimethylammonium chloride, and 2-acryloxyethyltrimethylammonium methyl sulfate).

Other monomers that can provide positively charged groups to the polymer include the dialkylaminoalkylamine adducts of alkenylazlactones (e.g., 2-(diethylamino)ethylamine, (2-aminoethyl)trimethylammonium chloride, and 3-(dimethylamino)propylamine adducts of vinyldimethylazlactone) and diallylamine monomers (e.g., diallylammonium chloride and diallyldimethylammonium chloride).

In some preferred embodiments, the optional hydrophilic monomer may have an ethylenically unsaturated group such as a (meth)acryloyl group and a poly(alkylene oxide) group. For example, the hydrophilic monomer can be a poly(alkylene oxide) mono(meth)acrylate compounds, where the terminus is a hydroxy group, or an alkyl ether group. Such monomers are of the general Formula (VI).

R⁹—O—(CH(R⁹)—CH₂—O)ₚ—C(O)—C(R⁹)═CH₂ (VI)

In Formula (VI), each R⁹ is independently hydrogen or a C₁-C₄ alkyl. The variable p is at least 2 such as, for example, 2 to 100, 2 to 50, 2 to 20, or 2 to 10.

In one embodiment, the poly(alkylene oxide) group (depicted as —(CH(R⁹)—CH₂—O)ₚ—) is a poly(ethylene oxide). In another embodiment, the poly(alkylene oxide) group is a poly(ethylene oxide-co-propylene oxide). Such copolymers may be block copolymers, random copolymers, or gradient copolymers.

Other representative examples of suitable hydrophilic monomers include but are not limited to acrylic acid; methacrylic acid; 2-acrylamido-2-methyl-1-propanesulfonic acid; 2-hydroxyethyl (meth)acrylate; N-vinylpyrrolidone; N-vinylcaprolactam; acrylamide; mono- or di-N-alkyl substituted acrylamide; t-butyl acrylamide; dimethylacrylamide; N-octyl acrylamide; poly(alkoxyalkyl) (meth)acrylates including 2-(2-ethoxyethoxy)ethyl (meth)acrylate, 2-ethoxyethyl (meth)acrylate, 2-methoxyethoxyethyl (meth)acrylate, 2-methoxyethyl methacrylate, polyethylene glycol mono(meth)acrylates; alkyl vinyl ethers, including vinyl methyl ether; and mixtures thereof. Preferred hydrophilic monomers include those selected from the group consisting of dimethylacrylamide, 2-hydroxyethyl (meth)acrylate, and N-vinylpyrrolidinone.

In some embodiments, the monomer composition used to form the carbonyl-containing polymer can optionally include a hydrophobic monomer. As used herein, the term “hydrophobic monomer” refers monomers having a water miscibility (water in monomer) that is less than 1 weight percent. The hydrophobic monomers can be used in amounts that do not deleteriously affect the binding performance of the guanidinyl-containing monomer polymer and/or the water dispersibility of the guanidinyl-containing polymer. When present, the hydrophobic monomer is typically present in an amount in a range of 1 to 20 weight percent, 1 to 10 weight percent, or 1 to 5 weight percent based on a total weight of monomers in the monomer composition.

Useful classes of hydrophobic monomers include alkyl acrylate esters and amides, exemplified by straight-chain, cyclic, and branched-chain isomers of alkyl esters containing C₁-C₃₀ alkyl groups and mono- or dialkyl acrylamides containing C₁-C₃₀ alkyl groups. Useful specific examples of alkyl acrylate esters include: methyl acrylate, ethyl acrylate, n-propyl acrylate, n-butyl acrylate, iso-amyl acrylate, n-hexyl acrylate, n-heptyl acrylate, isobornyl acrylate, n-octyl acrylate, iso-octyl acrylate, 2-ethylhexyl acrylate, iso-nonyl acrylate, decyl acrylate, undecyl acrylate, dodecyl acrylate, lauryl acrylate, tridecyl acrylate, and tetradecyl acrylate. Useful specific examples of alkyl acrylamides include mono- and diacrylamides having pentyl, hexyl, heptyl, isobornyl, octyl, 2-ethylhexyl, iso-nonyl, decyl, undecyl, dodecyl, tridecyl, and tetradecyl groups may be used. The corresponding methacrylate esters may be used.

Additional useful classes of hydrophobic monomers further include vinyl monomers such as vinyl acetate, styrenes, and alkyl vinyl ethers, and maleic anhydride.

The monomer composition used to form the carbonyl-containing polymer is typically combined with a free radical initiator to form the polymerized product. Any suitable free radical initiator can be used. The initiator is typically present in an amount in the range of 0.01 to 5 weight percent, in the range of 0.01 to 2 weight percent, in the range of 0.01 to 1 weight percent, or in the range of 0.01 to 0.5 weight percent based on a total weight of monomers in the monomer composition.

In some embodiments, a thermal initiator can be used. Thermal initiators can be water-soluble or water-insoluble (i.e., oil-soluble) depending on the particular polymerization method used. Suitable water-soluble initiators include, but are not limited to, persulfates such as potassium persulfate, ammonium persulfate, sodium persulfate, and mixtures thereof; an oxidation-reduction initiator such as the reaction product of a persulfate and a reducing agent such as a metabisulfite (e.g., sodium metabisulfite) or a bisulfate (e.g., sodium bisulfate); or 4,4′-azobis(4-cyanopentanoic acid) and its soluble salts (e.g., sodium or potassium). Suitable oil-soluble initiators include, but are not limited to, various azo compound such as those commercially available under the trade designation VAZO from DuPont (Wilmington, Del., USA) including VAZO 67, which is 2,2′-azobis(2-methylbutane nitrile), VAZO 64, which is 2,2′-azobis(isobutyronitrile), and VAZO 52, which is (2,2′-azobis(2,4-dimethylpentanenitrile); and various peroxides such as benzoyl peroxide, cyclohexane peroxide, lauroyl peroxide, and mixtures thereof.

In many embodiments, a photoinitiator can be used. Some illustrative photoinitiators are benzoin ethers (e.g., benzoin methyl ether or benzoin isopropyl ether) or substituted benzoin ethers (e.g., anisoin methyl ether). Other exemplary photoinitiators are substituted acetophenones such as 2,2-diethoxyacetophenone or 2,2-dimethoxy-2-phenylacetophenone (commercially available under the trade designation IRGACURE 651 from BASF Corp. (Florham Park, N.J., USA) or under the trade designation ESACURE KB-1 from Sartomer (Exton, Pa., USA)). Still other exemplary photoinitiators are substituted alpha-ketols such as 2-methyl-2-hydroxypropiophenone, aromatic sulfonyl chlorides such as 2-naphthalenesulfonyl chloride, and photoactive oximes such as 1-phenyl-1,2-propanedione-2-(0-ethoxycarbonyl)oxime. Other suitable photoinitiators include, for example, 1-hydroxycyclohexyl phenyl ketone (IRGACURE 184), bis(2,4,6-trimethylbenzoyl)phenylphosphineoxide (IRGACURE 819), 1-[4-(2-hydroxyethoxy)phenyl]-2-hydroxy-2-methyl-1-propane-1-one (IRGACURE 2959), 2-benzyl-2-dimethylamino-1-(4-morpholinophenyl)butanone (IRGACURE 369), 2-methyl-1-[4-(methylthio)phenyl]-2-morpholinopropan-1-one (IRGACURE 907), and 2-hydroxy-2-methyl-1-phenyl propan-1-one (DAROCUR 1 173).

The guanidinyl-containing polymers according to Formula (IV) are often the reaction product of a carbonyl-containing polymer precursor and a guanylating agent of Formula (VII).

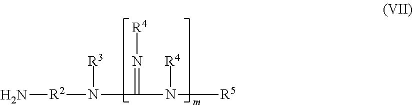

In Formula (VII), the group R² is a covalent bond, C₂-C₁₂ (hetero)alkylene, or C₅-C₁₂ (hetero)arylene. Group R³ is hydrogen, C₁-C₁₂ (hetero)alkyl, or C₅-C₁₂ (hetero)aryl. Each R⁴ is independently hydrogen, C₁-C₁₂ (hetero)alkyl, or C₅-C₁₂ (hetero)aryl. Group R⁵ is H, C₁-C₁₂ (hetero)alkyl, or C₅-C₁₂ (hetero)aryl, or —N(R⁴)₂. The variable m is equal to 1 or 2.

For ease of description, the carbonyl-containing polymer can be represented by the formula Polymer-C(═O)—R. The carbonyl group can be in the backbone or in a pendant group but is usually in a pendant group. When reacted with a guanylating agent of Formula (VII), the carbonyl group in the carbonyl-containing polymer undergoes a condensation reaction with a terminal amine group of the guanylating agent. The guanidinyl-containing polymer typically has guanidinyl-containing pendant groups of Formula (VIII).

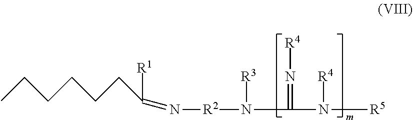

The groups R², R³, R⁴, and R⁵ are the same as described above for Formula (VII). The group of formula

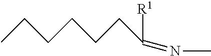

in Formula (VIII) is the linkage formed between the terminal amine of the ligand compound of Formula (VII) and the carbonyl group of the carbonyl-containing polymer. The wavy line denotes the attachment site of the group via a covalent bond to the rest of the polymer. Group R¹ is hydrogen (when the carbonyl group is an aldehyde group), C₁-C₁₂ (hetero)alkyl (when the carbonyl group is a ketone group and the ketone group is part of a pendant group), or C₅-C₁₂ (hetero)aryl (when the carbonyl group is a ketone group and the ketone group is part of a pendant group), or a residue of the polymer chain (when the carbonyl group is a group in the backbone of the carbonyl-containing polymer). In most embodiments, the group of Formula (VIII) is part of a pendant group of the guanidinyl-containing polymer.

In other embodiments, the guanidyl-containing polymer may be prepared in which the imine linking group (—C(R¹)═N—) is reduced to an amine linking group (—CH(R¹)—NH—). This may be effected by treating the extant ligand functional polymer with a reducing agent, such as sodium cyanoborohydride, or the reduction may be effected in situ by adding the reducing agent to the reaction mixture of the carbonyl functional (co)polymer and the compound of Formula V.

In many embodiments, some but not all of the carbonyl groups of the carbonyl-containing polymer are reacted with the guanylating agent of Formula (VII). Typically, at least 0.1 mole percent, at least 0.5 mole percent, at least 1 mole percent, at least 2 mole percent, at least 10 mole percent, at least 20 mole percent, or at least 50 mole percent of the carbonyl groups in the carbonyl-containing polymer precursor are reacted with the guanylating agent. Up to 100 mole percent, up to 90 mole percent, up to 80 mole percent, or up to 60 mole percent of the carbonyl groups can be reacted with the guanylating agent. For example, the guanylating agent can be used in amounts sufficient to functionalize 0.1 to 100 mole percent, 0.5 to 100 mole percent, 1 to 90 mole percent, 1 to 80 mole percent, 1 to 60 mole percent, 2 to 50 mole percent, 2 to 25 mole percent, or 2 to 10 mole percent of the carbonyl groups in the carbonyl-containing polymer.

The guanidinyl-containing polymer can be crosslinked. In some embodiments, the carbonyl-containing polymer is crosslinked prior to reaction with the guanylating agent. The carbonyl-containing polymer can be crosslinked either by addition of a crosslinking monomer in the monomer composition used to form the carbonyl-containing polymer or by reaction of some of the carbonyl groups of the previously formed carbonyl-containing polymer with a suitable crosslinking agent. In other embodiments, crosslinking can occur after reaction of the carbonyl-containing polymer with the guanylating agent. In this embodiment, crosslinking can occur by reaction of some of the remaining carbonyl groups (those carbonyl groups in the carbonyl-containing polymer precursor that were not reacted in the process of forming the guanidinyl-containing polymer) with a suitable crosslinking agent or by reaction of some of the guanidinyl groups with a crosslinking agent.

Suitable crosslinking monomers for use in the monomer composition to form the carbonyl-containing polymer include, but are not limited to, N,N′-(hetero)alkylenebis(meth)acrylamide. These crosslinking monomers have at least two (meth)acryloyl groups that can react to crosslink one polymeric chain with another polymeric chain or that can react to crosslink one part of a polymeric chain with another part of the same polymeric chain. Suitable N,N′-alkylenebis(meth)acrylamide crosslinking monomers include, but are not limited to, those having an alkylene group with 1 to 10, 1 to 8, 1 to 6, or 1 to 4 carbon atoms such as N,N′-methylenebisacrylamide, N,N′-methylenebismethacrylamide, N,N′-ethylenebisacrylamide, N,N′-ethylenebismethacrylamide, N,N′-propylenebisacrylamide, N,N′-propylenebismethacrylamide, N,N′-hexamethylenebisacrylamide, and N,N′-hexamethylenebismethacrylamide. Suitable N,N′-heteroalkylenebis(meth)acrylamide crosslinking monomers include, but are not limited to, N,N′-cystaminebisacrylamide, N,N′-piperazinebisacrylamide, and N,N′-piperazinebismethacrylamide. These crosslinking monomers are commercially available from various suppliers such as Sigma-Aldrich (Milwaukee, Wis.) and Polysciences, Inc. (Warrington, Pa.). Alternatively, these crosslinking monomers can be synthesized by procedures described in the art such as, for example, in Rasmussen, et al., Reactive Polymers, 16, 199-212 (1991/1992).

Suitable crosslinkers for reaction with carbonyl groups of the carbonyl-containing polymer precursor or remaining carbonyl groups of the guanidinyl-containing polymer include molecules comprising two or more amine, hydrazine, hydrazide, or O-substituted hydroxylamine moieties. Specific examples of polyamine (compounds with two or more amine groups) crosslinkers include 1,2-ethanediamine, 1,2-propanediamine, 1,3-propanediamine, 1,6-hexanediamine, tris-(2-aminoethyl)amine, diethylenetriamine, triethylenetetraamine, tetraethylenepentaamine, N,N′-bis(3-aminopropyl)piperazine, N-(2-aminoethyl)piperazine, polyethyleneimine, polyallylamine, and the like. Specific examples of polyhydrazines (compounds with two or more hydrazine groups) include I,Γ-ethylenebishydrazine, 1,1′-propylenebishydrazine, 1,1′-ethylenebis(1-cyclohexylhydrazine), 1,1′-decamethylenebis(1-n-butylhydrazine), and the like. Specific examples of useful polyhydrazides (compounds with two or more hydrazide groups) include succinic dihydrazide, adipic dihydrazide, terephthalic dihydrazide, 1,3-diaminoguanidine, and the like. Specific examples of polyhydroxylamines (compounds with two or more O-substituted hydroxylamine groups) include O,O′-ethylenebishydroxylamine (1,2-bisaminoxyethane), 1,6-bisaminoxyhexane, and the like. Alternatively, crosslinkers comprising two or more different moieties selected from amine, hydrazine, hydrazide, or O-substituted hydroxylamine moieties can be used.

Suitable crosslinkers for reaction with the guanidinyl groups of the guanidinyl-containing polymer include amine-reactive compounds such as bis- and polyaldehydes such as glutaraldehyde, bis- and polyepoxides such as butanedioldiglycidylether and ethyleneglycoldiglycidylether, polycarboxylic acids and their derivatives (e.g., acid chlorides), polyisocyanates, formaldehyde-based crosslinkers such as hydroxymethyl and alkoxymethyl functional crosslinkers, such as those derived from urea or melamine.

Rather than reacting a precursor polymer with a guanylating agent to prepare a guanidinyl-containing polymer, the guanidinyl-containing polymer can be prepared by free radical polymerization of a guanidinyl-containing monomer, which refers to a monomer having an ethylenically unsaturated group and a guanidinyl-containing group. Example guanidinyl-containing monomers are of Formula (IX) and (X).

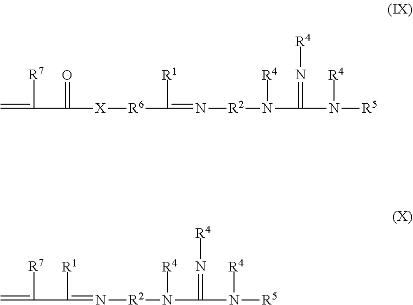

In Formulas (IX) and (X), group R¹ is hydrogen, C₁-C₁₂ alkyl, or C₅-C₁₂ (hetero)aryl. Group R² is a covalent bond, a C₂ to C₁₂ alkylene, a C₅-C₁₂ (hetero)arylene, a divalent group of formula

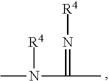

or a divalent group of formula

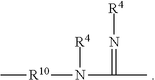

Group R¹⁰ is C₂ to C₁₂ alkylene, or C₅-C₁₂ (hetero)arylene. Each R³ is independently hydrogen, hydroxyl, C₁-C₁₂ alkyl, or C₅-C₁₂ (hetero)aryl. R³ is preferably hydrogen or C₁-C₄ alkyl. Group R⁴ is hydrogen, C₁-C₁₂ alkyl, C₅-C₁₂ (hetero)aryl, or —N(R³)₂. Preferably, R⁴ is hydrogen or C₁-C₄ alkyl. Group X is oxy or —NR³—. Group R⁶ is a C₂ to C₁₂ alkylene. Group R⁷ is hydrogen or CH₃.

The monomers of Formula (IX) and (X) can be formed, for example, by a condensation reaction of a carbonyl-containing monomer with the guanylating agent of Formula (VII). Example carbonyl-containing monomers include, but are not limited to, acrolein, vinyl methyl ketone, vinyl ethyl ketone, vinyl isobutyl ketone, isopropenyl methyl ketone, vinyl phenyl ketone, diacetone (meth)acrylamide, acetonyl acrylate, and acetoacetoxyethyl (meth)acrylate.

The monomers of Formula (IX) or (X) may be reacted to form homopolymers or can be copolymerized with other ethylenically unsaturated monomers such as any of the hydrophilic monomers described above. A free radical initiator such as those described above in the preparation of the carbonyl-containing polymer can be used. This reaction is further described in International Patent Publication WO 2011/103106 A1 (Rasmussen et al.).

Guanidinyl-containing polymers formed from a monomer of Formula (X) or (XI) are typically crosslinked by addition of a crosslinking monomer to the monomer composition. Suitable crosslinking monomers include N,N′-alkylenebis(meth)acrylamide, N,N′-heteroalkylenebis(meth)acrylamide, or a combination thereof. More specific crosslinkers are the same as described above for use in a monomer composition for preparation of the carbonyl-containing polymers. Alternatively, the guanidinyl-containing polymers can be formed without a crosslinking monomer and the guanidinyl groups can be reacted with crosslinkers as described above.

The guanidinyl-containing polymer can be bound to the substrate using any suitable method or means. In some embodiments, the guanidinyl-containing polymer is grafted (i.e., covalently attached) to the substrate. In other embodiments, the guanidinyl-containing polymer is contacted with the substrate prior to crosslinking and is crosslinked in the presence of the substrate. When the substrate includes fibers (e.g., the substrate includes a woven or nonwoven fabric), the crosslinked guanidinyl-containing polymer can surround fibers. The fibers and the crosslinked guanidinyl-containing polymers can be so intermingled that separation is not possible by a technique such as peeling or dissolution or by any other technique without the destruction of the wipe.

The cationic coating composition that includes the guanidinyl-containing polymer is applied to the substrate. Coating methods include the techniques commonly known such as dip, spray, knife, bar, slot, slide, die, roll, and gravure coating. The cationic coating can be disposed on a surface of the substrate or distributed throughout the substrate. For example, the cationic coating composition can be applied to the surface of the substrate. Depending on the porosity of the substrate, the viscosity of the cationic coating composition, and the relative volume of the cationic coating composition to that of the substrate, at least some of the cationic coating composition can permeate into the substrate. In some examples, the cationic coating can be poured over the substrate such that the substrate is immersed in or covered with the cationic coating composition. The cationic coating composition often includes a liquid such as water, an organic solvent such as a polar organic solvent (e.g., a solvent that is miscible with water), or a mixture thereof. The cationic coating composition can additionally include the crosslinking agent for the guanidinyl-containing polymer. Depending on the chemistry used to bind the guanidinyl-containing polymer to the substrate, a compound for grafting or attaching the guanidinyl-containing polymer to the substrate can be included in the cationic coating composition. After application to the substrate, the cationic coating composition can be dried to remove the liquid or any desired portion of the liquid. In some embodiments, the drying to remove the liquid is accomplished through evaporation.

In some embodiments, the cationic coating composition is applied to the substrate by first applying the precursor polymer for the guanidinyl-containing polymer followed by application of the guanylating agent. For example, an amino-containing polymer precursor or a carbonyl-containing polymer precursor can be applied to the substrate in a first coating composition. A second coating composition can then be applied that includes the guanylating agent. The crosslinking agent can be added in the first coating composition with the precursor polymer, in the second coating composition with the guanylating agent, or in a third coating composition. Any of the coating compositions can include an optional compound for grafting the guanidinyl-containing polymer to the substrate.

In other embodiments, the guanidinyl-containing polymer is applied to the substrate. The coating composition that contains the guanidinyl-containing polymer can further include a crosslinking agent, an optional grafting compound, or a mixture thereof. Alternatively, the crosslinking agent and/or optional grafting agent can be added in a second coating composition.

Some substrates have amine-reactive functional groups such as halide groups, epoxy groups, ester groups, or isocyanate groups. These amine-reactive groups can react with amino groups of the guanidinyl-containing polymer. The amino groups can be part of the guanidinyl group (such as a terminal amino group) or any other amino groups that are present in the guanidinyl-containing polymer. For example, if the guanidinyl-containing polymer was formed from an amino-containing polymer precursor, there can be amino groups in the backbone of the guanidinyl-containing polymer.

The amine-reactive functional groups on the substrate may be part of the polymeric material used to form the substrate or may be provided by any of the techniques known to one in the art. In one embodiment, the substrate may have a primer layer containing a polymer having amine-reactive functional groups. That is, the substrate includes a base polymer layer and a primer layer. Especially useful polymers of use in the primer layer are azlactone functional polymers such as those described in U.S. Pat. No. 7,101,621 (Haddad et al.). Such primer layer coatings are typically hydrophilic and are compatible with the cationic coating composition. Useful coating techniques for the primer layer include applying a solution or dispersion of the polymer having amine-reactive functional groups, optionally further including a crosslinker, onto the substrate. Coating methods include the techniques commonly known such as dip, spray, knife, bar, slot, slide, die, roll, and gravure coating. The application step is generally followed by evaporating the solvent to form the polymer coating.

In some embodiments, the polymer having amine-reactive functional groups may be grafted to the surface of a substrate by ionizing radiation-initiated graft polymerization of a monomer having a free-radically polymerizable group and a second functional group reactive with the guanidinyl-containing polymer. One such polymer having an amine-reactive functional group is described U.S. Pat. No. 8,551,894 (Seshadri et al.). Suitable monomers include, for example, an azlactone-functional monomer, isocyanatoethyl (meth)acrylate, and a glycidyl (meth)acrylate. Other suitable monomers include, for example, those having a carbonyl group as described in U.S. Pat. No. 8,377,672 (Rasmussen et al.). The monomers can graft (i.e., form a covalent bond) to the surface of the substrate when exposed to an ionizing radiation, preferably e-beam or gamma radiation. That is, reaction of an ethylenically unsaturated group (e.g., a (meth)acryloyl group) of the monomer with the surface of the substrate in the presence of the ionizing radiation results in grafting to the substrate via the ethylenically unsaturated group.

Some substrates have carbonyl-reactive groups such as amines. These carbonyl-reactive groups can react with a carbonyl-containing polymer precursor prior to reaction with the guanylating agent or can react with any residual carbonyl groups in the guanidinyl-containing polymer after reaction with the guanylating agent.

The carbonyl-reactive functional groups on the substrate may be part of the polymeric material used to form the substrate or may be provided by any of the techniques known to one in the art. In some embodiments, the carbonyl-reactive groups can be grafted to the surface of a substrate by ionizing radiation-initiated graft polymerization of a monomer having a free-radically polymerizable group and a second group capable of reacting with a carbonyl group of either the carbonyl-containing precursor or any residual carbonyl groups in the guanidinyl-containing polymer after reaction with a guanylating agent. Such monomers are various amino-containing monomers such as those of Formula (V) where R⁸ is hydrogen.

In another method of bonding the guanidinyl-containing polymer to the substrate, a compound such as benzophenone or acetophenone can be added to the monomer composition used to form the carbonyl-containing precursor. Upon exposure to UV radiation, the benzophenone or acetophenone can abstract a hydrogen atom from the polymeric material of the substrate. This abstraction results in the formation of a free radical site on the polymeric material of the substrate. The monomers then interact with the free radical site and become graft polymerized onto the substrate. The covalently attached carbonyl-containing polymer can then be treated with a guanylating agent to form the guanidinyl-containing polymer.

The bonding of the guanidinyl-containing polymer to the substrate provides enhanced affinity for various microorganisms while retaining many of the desirable features of the substrate such as mechanical stability, thermal stability, porosity, and flexibility. The wipes typically contain an amount of the bound guanidinyl-containing polymer in a range of 0.1 to 10 weight percent, in a range of 0.1 weight percent to 5 weight percent, in a range of 0.1 to 3 weight percent, in a range of 0.1 to 2, or in a range of 0.1 weight percent to 1 weight percent, based on a total weight of the wipe.

Further details related to guanidinyl containing polymers can be found, for example in WO 2014/209798, the disclosure of which is incorporated herein by reference thereto.

### Optional Components in First, Second, or Both Compositions

Disclosed compositions (first, second or both) can also include one or more other optional components. For example surfactants including anionic surfactants, amphoteric surfactants, nonionic surfactants or combinations thereof may be included in disclosed compositions. Surfactants, if utilized, may be used to stabilize the dispersed particles in the composition for example. In some embodiments, anionic surfactants or nonionic surfactants can optionally be utilized in disclosed compositions.

Anionic surfactants can include, but are not limited to, sarcosinates, glutamates, alkyl sulfates, sodium or potassium alkyleth sulfates, ammonium alkyleth sulfates, ammonium laureth-n-sulfates, laureth-n-sulfates, isethionates, alkyl and aralkyl glycerylether sulfonates, alkyl and aralkyl sulfosuccinates, alkylglyceryl ether sulfonates, alkyl phosphates, aralkyl phosphates, alkylphosphonates, and aralkylphosphonates. These anionic surfactants may have a metal or organic ammonium counterion.

In some embodiments anionic surfactants selected from sulfonates and sulfates, and phosphonates and phosphates may be utilized in disclosed compositions.

Suitable anionic surfactants can include sulfonates and sulfates such as alkyl sulfates, alkylether sulfates, alkyl sulfonates, alkylether sulfonates, alkylbenzene sufonates, alkylbenzene ether sulfates, alkylsulfoacetates, secondary alkane sulfonates, secondary alkylsulfates, and the like. Many of these can be represented by the formulas: R¹⁴—(OCH₂CH₂)ₙ(OCH(CH₃)CH₂)ₚ—(Ph)ₐ—(OCH₂CH₂)ₘ—(O)_{b}—SO₃-M+ and R¹⁴—CH[SO₃-M+]—R¹⁵ wherein: a and b=0 or 1; n, p, and m=0-100 (in some embodiments 0-20, and in some embodiments 0-10); R¹⁴ and R¹⁵ are (C₁-C₁₂)alkyl group (saturated straight, branched, or cyclic group) that may be optionally substituted by N, O, or S atoms or hydroxyl, carboxyl, amide, or amine groups provided at least one R¹⁴ or R¹⁵ is at least C₈; Ph=phenyl; and M is a cationic counterion such as H, Na, K, Li, ammonium, or a protonated tertiary amine such as triethanolamine or a quaternary ammonium group.

In the formula above, the ethylene oxide groups (i.e., the “n” and “m” groups) and propylene oxide groups (i.e., the “p” groups) can occur in reverse order as well as in a random, sequential, or block arrangement. In some embodiments for this class, R¹⁴ can include an alkylamide group such as R¹⁶⁻C(O)N(CH₃)CH₂CH₂— as well as ester groups such as —OC(O)—CH₂— wherein R¹⁶ is a (C₅-C₂₂)alkyl group (branched, straight, or cyclic group). Examples include, but are not limited to: alkyl ether sulfonates such as lauryl ether sulfates such as POLYSTEP B12 (n=3-4, M=sodium) and B22 (n=12, M=ammonium) available from Stepan Company, Northfield, Ill. and sodium methyl taurate (available under the trade designation NIKKOL CMT30 from Nikko Chemicals Co., Tokyo, Japan); secondary alkane sulfonates such as Hostapur SAS which is a sodium salt of a secondary alkane sulfonate.

Examples can include, for example (C₁₄-C₁₇) secondary alkane sulfonates (alpha-olefin sulfonates) available from Clariant Corp., Charlotte, N.C.; methyl-2-sulfoalkyl esters such as sodium methyl-2-sulfo(C₁₂₋₁₆)ester and disodium 2-sulfo(C₁₂-C₁₆)fatty acid available from Stepan Company under the trade designation ALPHASTEP PC-48; alkylsulfoacetates and alkylsulfosuccinates available as sodium laurylsulfoacetate (under the trade designation LANTHANOL LAL) and disodiumlaurethsulfosuccinate (STEPANMILD SL3), both from Stepan Company; alkylsulfates such as ammoniumlauryl sulfate commercially available under the trade designation STEPANOL AM from Stepan Company; dialkylsulfosuccinates such as dioctylsodiumsulfosuccinate available as Aerosol OT from Cytec Industries.

Suitable anionic surfactants can also include phosphates such as alkyl phosphates, alkylether phosphates, aralkylphosphates, and aralkylether phosphates. Many may be represented by the formula:

[R₁₄—(Ph)ₐ—O(CH₂CH₂O)n(CH₂CH(CH₃)O)ₚ]_{q}—P(O)[O-M+]ᵣ

wherein: Ph, R₁₄, a, n, p, and M are defined above; r is 0-2; and q=1-3; with the proviso that when q=1, r=2, and when q=2, r=1, and when q=3, r=0. As above, the ethylene oxide groups (i.e., the “n” groups) and propylene oxide groups (i.e., the “p” groups) can occur in reverse order as well as in a random, sequential, or block arrangement. Examples can include a mixture of mono-, di- and tri-(alkylalkoxylate)-o-phosphoric acid esters such as trilaureth-4-phosphate commercially available under the trade designation HOSTAPHAT 340KX from Clariant Corp., as well as PPG-5 ceteth 10 phosphate available under the trade designation CRODAPHOS SG from Croda Inc., Parsipanny, N.J., and mixtures thereof. Trade names for anionic surfactants include Rhodocal DS-10, Stepan Mild, and Complemix.

Amphoteric Surfactants. Surfactants of the amphoteric type include surfactants having tertiary amine groups, which may be protonated, as well as quaternary amine containing zwitterionic surfactants. In some embodiments ammonium carboxylates and ammonium sulfonates may be utilized.

The ammonium carboxylate class of surfactants can be represented by the following formula: R¹⁷—(C(O)—NH)a-R¹⁸—N+(R¹⁹)₂—R²⁰—COO— wherein: a=0 or 1; R¹⁷ is a (C₇-C₂₁)alkyl group (saturated straight, branched, or cyclic group), a (C₆-C₂₂)aryl group, or a (C₆-C₂₂)aralkyl or alkaryl group (saturated straight, branched, or cyclic alkyl group), wherein R¹⁷ may be optionally substituted with one or more N, O, or S atoms, or one or more hydroxyl, carboxyl, amide, or amine groups; R¹⁹ is H or a (C₁-C₈)alkyl group (saturated straight, branched, or cyclic group), wherein R¹⁹ may be optionally substituted with one or more N, O, or S atoms, or one or more hydroxyl, carboxyl, amine groups, a (C₆-C₉)aryl group, or a (C₆-C₉)aralkyl or alkaryl group; and R¹⁸ and R²⁰ are each independently a (C₁-C₁₀)alkylene group that may be the same or different and may be optionally substituted with one or more N, O, or S atoms, or one or more hydroxyl or amine groups. In some embodiments, in the formula above, R¹⁷ is a (C₁-Cis)alkyl group, R¹⁹ is a (C₁-C₂)alkyl group which can be substituted with a methyl or benzyl group and in some embodiments with a methyl group. When R¹⁹ is H it is understood that the surfactant at higher pH values could exist as a tertiary amine with a cationic counterion such as Na, K, Li, or a quaternary amine group. Examples of such amphoteric surfactants include, but are not limited to: certain betaines such as cocobetaine and cocamidopropyl betaine (commercially available under the trade designations MACKAM CB-35 and MACKAM L from Mclntyre Group Ltd., University Park, Ill.); monoacetates such as sodium lauroamphoacetate; diacetates such as disodium lauroamphoacetate; amino- and alkylamino-propionates such as lauraniinopropionic acid (commercially available under the trade designations MACKAM IL, MACKAM 2L, and MACKAM 15 IL, respectively, from Mclntyre Group Ltd.).

The ammonium sulfonate class of amphoteric surfactants are often referred to as “sultaines” or “sulfobetaines” and can be represented by the following formula: R¹⁷—(C(O)—NH)ₐ—R¹⁸—N+(R¹⁹)₂—R²⁰, —SO₃ wherein R¹⁷—R²⁰ and “a” are defined above. Examples include cocamidopropylhydroxysultaine (commercially available as MACKAM 50-SB from Mclntyre Group Ltd.). The sulfoamphoterics may be utilized instead of the carboxylate amphoterics in some embodiments since the sulfonate group will remain ionized at much lower pH values.

Nonionic Surfactants. Exemplary nonionic surfactants include, but are not limited to, alkyl glucosides, alkyl polyglucosides, polyhydroxy fatty acid amides, sucrose esters, esters of fatty acids and polyhydric alcohols, fatty acid alkanolamides, ethoxylated fatty acids, ethoxylated aliphatic acids, ethoxylated fatty alcohols (e.g., octyl phenoxy polyethoxyethanol available under the trade name TRITON X-100 and nonyl phenoxy poly(ethyleneoxy) ethanol available under the trade name NONIDET P-40, both from Sigma, St. Louis, Mo.), ethoxylated and/or propoxylated aliphatic alcohols (e.g., that available under the trade name Brij from ICI), ethoxylated glycerides, ethoxylated/propoxylated block copolymers such as the Pluronic and Tetronic surfactants available from BASF, ethoxylated cyclic ether adducts, ethoxylated amide and imidazoline adducts, ethoxylated amine adducts, ethoxylated mercaptan adducts, ethoxylated condensates with alkyl phenols, ethoxylated nitrogen-based hydrophobes, ethoxylated polyoxypropylenes, polymeric silicones, fluorinated surfactants (e.g., those available under the trade names FLUORAD-FS 300 from 3M Company, St. Paul, Minn., and ZONYL from Dupont de Nemours Co., Wilmington, Del.), and polymerizable (reactive) surfactants (e.g., SAM 211 (alkylene polyalkoxy sulfate) surfactant available under the trade name MAZON from PPG Industries, Inc., Pittsburgh, Pa.). In some embodiments, the nonionic surfactants useful in the compositions can be selected from the group consisting of Poloxamers such as PLURONIC from BASF, sorbitan fatty acid esters like TWEEN, and mixtures thereof.

Disclosed compositions can also include other optional components. One such optional component includes antimicrobial components. Cationic quaternary ammonium salts (some of which are antimicrobial agents) including, but are not limited to cetyl pyridinium chloride, cetrimonium bromide (CTAB), behentrimonium chloride, bis-biguanides include chlorhexidine salts and polymeric guanides such as polyhexamethylenebiguanide (PHMB), benzethonium chloride, chlorhexidine salts such as chlorhexidine gluconate, octenidine salts such as octenidine dihydrochloride, stearalkonium chloride etc. and mixtures thereof can be used. The antimicrobials, if present can generally be present from 0.01-1 wt % based on the total weight of the composition. Non-ionic antimicrobials such as triclosan can also be used. Cationic compounds can be used in relatively small concentrations, as long as the stability of the composition is not compromised.

Amine compounds can also optionally be added to disclosed compositions. These can include, for example ethoxylated amines such as Jeffamines, and peg 8 oleyl amine.

Humectants can also optionally be added to disclosed compositions. Suitable humectants may include for example glycerin, propylene glycol, sorbitol, polypropylene glycol, polyethylene glycol, and combinations thereof.

Skin conditioning agents including emollients and polymers can also optionally be added to disclosed compositions. Useful skin conditioning agents such as mono-, di- and tri-glycerides, castor oil, allantoin, lanolin and its derivatives, cetyl alcohol and cationic polymers could be utilized for example. Emollients may be selected from those in U.S. Pat. No. 5,951,993 which is incorporated herein by reference, for example.

Thickeners can also optionally be added to disclosed compositions. In some embodiments, the thickeners could be organic thickeners. Suitable organic thickeners can include for example guar gum, hydroxyethyl cellulose, hydroxyl propyl cellulose, hydroxybutyl cellulose, hydroxypropyl methyl cellulose, polyvinylpyrrolidinone, as well as those disclosed in U.S. Pat. No. 8,062,649, incorporated herein by reference, which are used in the amount typically ranging from 0.1-2 wt % based on the total weight of the composition. Inorganic thickeners such as hydrated silica may be used in the amount of about 0.5-10 wt % or greater based on the total weight of the composition.

In some embodiments one or both of the first and second compositions can include thickeners. In some embodiments one or both of the compositions can include not greater than 0.2 wt % thickener, not greater than 0.1 wt % thickener, or even not greater than 0.05 wt % thickener based on the total weight of the composition. In some embodiments the first composition can include a thickener in an amount not greater than 0.3 wt % thickener, not greater than 0.2 wt % thickener, not greater than 0.1 wt % thickener, or even not greater than 0.05 wt % thickener based on the total weight of the first composition.

Abrasive or mechanical exfoliating agents could also optionally be included in disclosed compositions. In some embodiments abrasive or exfoliating materials could include water insoluble abrasives such as phosphates, carbonates, silicates, hydrated silica, hydrated alumina, bentonite, as well as polymeric beads such a poly methyl methacrylate (PMMA), polystyrene, and polyolefin beads and particulates and the like as well as mixtures thereof. In some cases, other mild exfoliating agents could optionally be used in disclosed compositions for mechanically removing spores. Illustrative exfoliating agents could include arrowroot powder or walnut powder for example.

### Methods and Illustrative Steps Thereof

Disclosed methods can be utilized on virtually any surface including for example skin, medical equipment, surfaces in a medical environment, or any combinations thereof. In some embodiments, disclosed methods can be utilized on skin of patients, health care workers, other individuals, or any combination thereof. Both the first and the second compositions are liquids.

The surface on which the spores are located and are to be dislodged from can be contacted with a first composition by applying or bringing the first composition to the surface, by bringing the surface into contact with the first composition, or by combinations thereof. More specifically, the first composition and the surface can be brought into contact by spraying the first composition onto the surface, by dispensing the first composition onto the surface, by dipping the surface into the first composition, by pouring the first composition onto the surface, or by any combination thereof.

In some embodiments bringing the surface into contact with the first composition can be accomplished by dispensing the first composition into or onto an article. Dispensing can be accomplished via pouring, spraying, bringing the article into the first composition (e.g., dipping), or submerging the article in the first composition, for example. As indicated, the first composition can be dispensed into or onto an article.

In embodiments where the first composition is dispensed into an article, illustrative articles can include for example basins, bowls, and tubs. Such methods can be useful in instances where the skin from which the spores are to be removed is to be brought into contact with the first composition in the article, for example. More specifically, this can be useful if some part of, or all of the patient is going to be immersed in the first composition in the article. For example, the patient could be going to bathe in the article (e.g., a tub). Another example could include a basin where some part of a patient, for example one or more hands are to be immersed in the first composition in the basin. This can also be useful if a secondary article is going to be immersed in the first composition in the article and then that secondary article is going to be brought into contact with the skin.

The amount of the first composition dispensed into an article, or an effective amount, can depend at least in part on how the skin from which the spores are to be removed is to be brought into contact with the first composition in the article, the particular skin to be cleaned, the type of optional mechanical action (discussed below), whether or not there is a secondary article, or combinations thereof.

In some embodiments where the first composition is to be dispensed into an article, not less than 5 milliliters (mL) of the first composition can be dispensed into an article, not less than 1 mL of the first composition, or not less than 2 mL of the first composition. Relevant upper amounts of the first composition would depend at least in part on the particular article (e.g., its maximum volume), the volume to be immersed (if immersion is relevant) in the article, or combinations thereof.

In some embodiments where the composition is to be dispensed into an article, the amount dispensed, or an effective amount can also depend, at least in part, on the surface area, for example surface area of skin from which spores are to be removed. In some embodiments, not less than 1 mL of the first composition/10 cm2 of skin surface from which spores are to be removed can be dispensed into the article, and in some embodiments not less than 1 mL of the first composition/50 cm2 of skin surface from which spores are to be removed can be dispensed into the article.

In some embodiments, the step of contacting the surface with the first composition can include contacting the surface with an article that has been previously contacted with the first composition. For example, an article could be contacted with or treated with the composition and then that treated article could be contacted with the surface.

In some embodiments where the first composition is dispensed onto an article, illustrative articles can include for example wipes, sponges, cloths, loofahs, brushes, pads, or fibrous mats for example. The second composition is also (also when the first composition is also optionally dispensed onto an article) dispensed onto an article and discussion related to such articles (which contain the first composition or the second composition, or both in separate articles) can apply to the first composition or articles associated therewith, the second composition or articles associated therewith or both.

It should be understood that dispensing a composition onto an article can imply bringing the composition to the article, bringing the article to the composition, or any combination thereof. Such methods can be useful in instances where the skin from which the spores are to be removed is to be brought into contact with an article that is associated with the composition, for example. More specifically, this can be useful for wiping affected skin of a patient with the article to which the composition has been dispensed, to remove spores from the skin of the patient. In some embodiments the composition can be dispensed onto a wipe with a coating, for example a cationic coating.

The amount of the composition dispensed onto an article, or an effective amount, can depend at least in part on the particular surface to be cleaned (e.g., skin), the total surface area of the surface (e.g., skin) to be cleaned, the type of optional mechanical action (discussed below), the particular type of article (e.g., the amount the article can absorb, hold, etc. because of size or chemical components) or combinations thereof.

In some embodiments where the composition is to be dispensed onto an article, not less than 3 mL of the composition can be dispensed onto an article, not less than 4 mL of the composition, not less than 5 mL of the composition, or not less than 2 mL of the composition. Relevant upper amounts of the composition would depend at least in part on the particular article (e.g., its surface area, the material thereof, its porosity, etc.), a desired level of “wetness” of the article, and combinations thereof.

In some embodiments where the composition is to be dispensed onto an article, the amount dispensed, or an effective amount can also depend, at least in part, on the surface area of skin from which spores are to be removed. In some embodiments, not less than 1 mL of the composition/10 cm² of skin surface from which spores are to be removed can be dispensed onto the article, and in some embodiments not less than 5 mL of the composition/10 cm² of skin surface from which spores are to be removed can be dispensed onto the article.

In some embodiments where the composition is to be dispensed onto an article, an effective amount is one which provides a sufficient amount of the composition in contact with the skin surface from which spores are to be removed. In some embodiments, an effective amount can be described with respect to the amount of the composition necessary to saturate the article. An article is “saturated” when the article is contacted with more of the composition than the article can hold and reasonable pressure is applied to the article to remove the excess. In articles that are easily wet, “saturation” can be determined substantially immediately after incorporation. In articles that are more hydrophobic, it may be necessary to expose the article to the composition for a longer period of time. A reasonable amount of pressure can be that applied with an average hand squeezing motion until no further liquid is seen dripping from the wipe. The amount of the composition retained in the article after pressure has been applied can be referred to as the saturation amount.

In some embodiments, an effective amount of the composition that can be dispensed onto an article can be an amount that renders the wipe (for example) as wet as possible so that the saturation amount is bypassed. In some embodiments, it may not be desirable to go below the saturation level, or not less than 5% below the saturation amount. In some embodiments, an effective amount of the composition that can be dispensed onto an article can be not greater than 40% above the saturation amount of the article. In some embodiments, an effective amount can be not greater than 20% above the saturation amount of the article, in some embodiments not greater than 15% above the saturation amount, and in some embodiments not greater than 5% above the saturation amount. In some embodiments, an effective amount of a composition can be one that makes the article as wet as possible while maintaining a useful article.

For the purposes of illustration only, a 4 inch×6 inch SONTARA® 8005, 100% PET (DuPont) wipe has a saturation amount of 3.5 g liquid, so illustrative effective amounts for such an article could include not greater than 4.9 g of liquid, not greater than 4.2 g liquid, not greater than 4.0 g liquid, not greater than 3.7 g liquid, and not less than 3.3 g liquid.

In some embodiments where the composition is dispensed onto an article, a different first step can be utilized. For example, in some embodiments, a first step in disclosed methods can include obtaining an article containing a composition. An article containing a composition is an article that is capable of carrying an effective amount of a composition distributed throughout the material of the article.

The step of obtaining an article containing a composition can be accomplished by contacting a carrier with a composition, such as those compositions described above. This step can be carried out as discussed above with respect to dispensing the composition onto an article, which in this case is the carrier. The carrier can be a wipe, or a sponge for example. Also as discussed above, the carrier can be dipped into the composition, the composition can be sprayed onto the carrier, the composition can be applied to the carrier, or any combination thereof.

The step of obtaining an article containing a composition can also alternatively be accomplished by obtaining a carrier pre-moistened with the composition. For example, one or more articles containing a composition can be packaged together in any type of air tight or re-sealable packaging, for example a foil pack, a plastic container, or any combination thereof.

Some disclosed methods include a step of subjecting the surface contacted with the composition to mechanical action. This can include the first composition, the second composition, or both. Virtually any type of mechanical action could be utilized in disclosed methods. Illustrative types of mechanical action can include, for example, rubbing the surface with some article (for example an article treated with the composition, or an article not treated with the first composition), moving or scraping an article across the surface, moving a surface contacted with the composition across another surface contacted with the composition, or any combination thereof. In some embodiments, mechanical action can include rubbing, wiping, scraping, or scouring the surface with the article treated with the composition. In some embodiments, mechanical action can include moving a first surface contacted with the composition across or over a second surface contacted with the composition. A specific example of such an embodiment can include rubbing two hands contacted with the composition together.

The step of subjecting the surface to mechanical action can occur for any amount of time. In some embodiments, the surface can be subjected to mechanical action for not less than 5 seconds, not less than 10 seconds, or not less than 20 seconds. In some embodiments, the surface can be subjected to mechanical action for not greater than 2 minutes, or not greater than 1 minute, for example.

In some embodiments, the steps of contacting the surface with a first composition and optionally subjecting the surface in contact with the first composition to mechanical action can occur with at least some overlap. For example, in some embodiments, while at least some of the first composition is being contacted with the surface, mechanical action can begin. Specifically, for example, while a hand, or hands, is being dipped into the first composition (or even soaked in the first composition), the hands can be rubbed together. Either (or both) of the steps of contacting the surface with the first composition or subjecting the surface to mechanical action can be repeated more than once in some embodiments.

In some embodiments, the step of subjecting the skin to mechanical action can be described by the force of the mechanical action. In some embodiments, the mechanical action on the skin can have a force of not less than 20 N.

In some embodiments where the second composition is dispensed onto an article, an additional step can be utilized. For example, in some embodiments, a step in disclosed methods can include obtaining a cationic coated article containing a second composition. A cationic coated article containing a second composition is a cationic coated article that is capable of carrying, carries, or both an effective amount of a second composition distributed throughout the material of the article.

The step of obtaining a cationic coated article containing a second composition can be accomplished by contacting a cationic coated carrier with a second composition, such as those compositions described above. This step can be carried out as discussed above with respect to dispensing the second composition onto a cationic coated article, which in this case is the carrier. The carrier can be a wipe, or a sponge for example that is coated with a disclosed cationic coating. Also as discussed above, the carrier can be dipped into the second composition, the second composition can be sprayed onto the carrier, the second composition can be applied to the carrier, or any combination thereof.

The step of obtaining a cationic coated article containing a second composition can also alternatively be accomplished by obtaining a cationic coated carrier pre-moistened with the second composition. For example, one or more cationic coated articles containing a second composition can be packaged together in any type of air tight or re-sealable packaging, for example a foil pack, a plastic container, or any combination thereof.

In some embodiments, disclosed methods can include a first step of contacting a first composition with a surface and a second step of contacting the surface with a cationic coated wipe and a second composition. In some embodiments, the first composition can include greater than or equal to 60 wt % of at least one alcohol or in some embodiments the second composition can include greater than or equal to 60 wt % of at least one alcohol but both compositions cannot include greater than or equal to 60 wt % of at least alcohol. In some embodiments, the first composition can include at least one alcohol or in some embodiments the second composition can include at least one alcohol but both compositions cannot include at least alcohol. In some embodiments the second composition can be in contact with a wipe and in some embodiments both the first and the second compositions can be in contact with a wipe. In some embodiments where both the first and the second composition are in contact with a wipe, only the wipe in contact with the second composition need be a cationic coated wipe or both the wipes in contact with the first and the second composition can be a cationic coated wipe. In some such embodiments, the wipes in contact with the first and second compositions may or may not be the same type of wipe (e.g., material of the wipe).

### EXAMPLES

Objects and advantages are further illustrated by the following examples, but the particular materials and amounts thereof recited in these examples, as well as other conditions and details, should not be construed to unduly limit this disclosure.

Table 1 describes the list of reagents utilized herein.

TABLE 1

| Component | Supplier | Chemical Composition |
|---|---|---|
| GPEI or G-PEI | 3M Company, St. Paul, | Synthesis described below |
| | MN | |
| BUDGE | TCI America, Portland | butanedioldiglycidylether |
| | OR | |
| Butyl GPEI | 3M Company, St. Paul, | Synthesis described below |
| Solution | MN | |
| Ultrez 20 | Lubrizol Advanced | Acrylic acid polymer |
| | Material, Wickliffe, OH | |
| D/E Neutralizing | Becton Dickinson, | Dey/Engley Neutralizing |
| Broth | Franklin Lakes, NJ | Broth |
| Dodecyl GPEI | 3M Company, St. Paul, | Synthesis described below |
| Solution | MN | |
| 200 Proof | Columbus Chemical | Ethanol |
| Ethanol | Industries, Columbus, | |
| | WI | |
| IPA | EMD Millipore, | Isopropyl alcohol |
| | Billerica, MA | |
| HCl | J. T. Baker of Avantor | hydrochloric acid |
| | Preformance Materials, | |
| | Center Valley, PA | |
| Octyl GPEI | 3M Company, St. Paul, | Synthesis described below |
| Solution | MN | |
| 5k PEG GPEI | 3M Company, St. Paul, | Synthesis described below |
| Solution | MN | |
| | Alfa Aesar, Ward Hill, | O-Methylisourea hemi- |
| | MA | sulfate |
| PEI | Polysciences, | Polyethylenimine, Catalog |
| | Warrington PA | # 00318, 70,000 MW, 30% |
| | | w/w solution in water |
| Tween 20 | Sigma-Aldrich Corp. St. | polyoxyethylene sorbitan |
| | Louis, MO | monolaurate |
| NaOH | EM Science of EMD | Sodium Hydroxide |
| | Millipore, Billerica, MA | |
| Sontara 8005 | Previously available | 100% PET nonwoven |
| wipe | from DuPont, | |
| | Wilmington, DE; | |
| | currently available from | |
| | Jacob Holm, Basel, | |
| | Switzerland | |
| Ahlstrom 200 | Previously available | 50% PET, 50% cellulose |
| wipe | from Ahlstrom, | nonwoven |
| | Helsinki, Finland, | |
| | currently available from | |
| | Suominen, Helsinki, | |
| | Finland | |
| VITRO-SKIN ® | IMS Inc., Portland ME | |
| substrate | | |
| Blood agar plates | Thermo Fisher | Trypticase soy agar plates |
| | Scientific, Waltham, | with 5% sheep's blood |
| | MA | |

### Preparation of Butyl, Octyl and Dodecyl GPEI Solutions

A polyethylenimine (PEI) of 70,000 MW from Polysciences Inc., Warrington, Pa. was used as starting material. Butyl GPEI (10 mole % of amine groups reacted with butyl bromide, then 25 mole % of amines guanylated) was prepared by procedures similar to those described in Examples 1 and 2 of WO2011/109151, the disclosure of which is incorporated herein by reference thereto. Octyl GPEI (10 mole % octyl bromide and 25 mole % guanylated) and dodecyl GPEI (20 mole % dodecyl bromide and 25 mole % guanylated) were prepared similarly.

### Preparation of 5K PEG GPEI Solutions

For preparation of the 5K PEG GPEI solution, the alkyl bromide (above) was replaced with a 5000 MW PEG epoxide, PGI-EP-5k, available from Nanocs, Inc., New York, N.Y., and used to react with 10 mole % of the amine groups by a similar procedure.

### Preparation of Formulations

All of the formulations that were tested for their ability to remove spores from Vitro Skin were constructed in the same fashion.

For water and surfactant solutions, the water was first added to a glass jar, then the surfactant was added and shaken briefly to mix.

For alcohol solutions, the water was first added to a glass jar, then, if applicable, a thickener (ULTREZ 20) was added and rolled at 45 rpm overnight (approximately 18 hours) until the thickener was dispersed in the solution. Once the thickener was dispersed, triethanolamine was added dropwise until the pH was approximately 7. In solutions which did not have a thickener the pH adjustment was not necessary. For all solutions alcohol was added as the final step and they were briefly shaken to mix.

### Preparation of Cationic Coated Wipes

### Preparation of a 25% Guanylated Polyethyleneimine (“G-PEI” or “GPEI”) Coated Nonwoven Wipe

Polyethylenimine, 70,000 MW (658.2 grams of a 30 wt. % solution in water, 4.59 amine equivalents) was charged to a 3 L 3-necked round bottom flask equipped with overhead stirring. O-methylisourea hemisulfate (141.2 grams, 1.15 equivalents) was charged to a 1 L beaker, and enough deionized water was added to bring the total weight to 652.8 grams. The contents of the beaker were stirred magnetically until all of the O-methylisourea hemisulfate dissolved, then the solution was poured into the round bottom flask. The reaction mixture was allowed to stir at ambient temperature overnight (about 22 hours).

Analysis by NMR spectroscopy indicated conversion to the desired product. Percent solids was determined using an Ohaus moisture balance (Model Number MB35, obtained from Ohaus Corp., Parsippany, N.J.), and found to be 25.3 wt. %.

A sample of the G-PEI solution prepared as above (19.79 grams of a 25.3 wt. % solids solution) was diluted to a total of 500 grams with deionized water and mixed thoroughly. In a second container BUDGE (2.35 grams) was diluted to a total of 500 grams with deionized water and mixed thoroughly.

The two solutions were combined and mixed thoroughly. An appropriate amount of the solution was pipetted onto an 8″ by 10″ sheet of the nonwoven material The solution volume used was 20 mL for Sontara 8005 and Ahlstrom 200 or 30 mL for Texel 100 The sheet was scrunched/massaged until the solution appeared to thoroughly wet the sheet. After the sheet was thoroughly wetted it was hung to dry overnight (approximately 18 hours).

Once dry several (from 1 to 4) coated sheets were submerged in 1 L of a 50/50 mixture of methanol and water which was shaken at 150 rpm on an Orbital shaker for about 30 minutes. After the 30 minutes the wipes were removed from the solution, squeezed to remove the excess solution and submerged in 1 L of DI water, after which they were again shaken at 150 rpm for about 30 minutes. This was repeated once more with fresh DI water before the wipes were hung up to dry overnight.

After drying, small pieces of the wipe were cut off and dipped into a 0.1% solution of tartrazine dye and shaken for 5 minutes. The pieces were removed and washed three times in fresh distilled water for 5 minutes per wash. Coated pieces were stained evenly an orangish-yellow even after rinsing, unlike uncoated pieces of the wipe. This indicated the presence of a uniform, crosslinked coating of the cationic polymer on the surfaces of the nonwoven.

General Description of In-Vitro Experiment Designed to Evaluate Ability of Test Formulations to Remove Spores from Skin Like Surfaces

The day before testing, 36 mm disks of VITRO-SKIN® substrates (IMS Inc., Portland, Me.) were punched out and put in a VITRO-SKIN® substrates hydration chamber containing a mixture made from 298 g of water and 52 g of glycerin (per manufacturer's instructions).

The VITRO-SKIN® substrate disks were left to hydrate for at least 24 hours before testing. On the day of testing, first 3*N+6 (N is the number of samples to be tested) 50 mL conical tubes were filled with 15 mL of D/E broth each.

Then the stainless steel plates (5″ by 7.125″, 24 GA) were lined up on the counter top and cleaned by first spraying with water and wiping dry with paper towels. Second the plates were sprayed with 70% IPA and wiped dry with paper towels. Once dry, two pieces of double sided tape (no longer than 2.5 cm) were placed in a cross pattern in the middle of the plates. One disk of hydrated VITRO-SKIN® substrate was placed onto the double sided tape and gently rubbed to ensure they would stick on the plates. Three pieces of VITRO-SKIN® substrates were used for each sample to be tested and three extra pieces were prepared as the recovery control.

The starting inoculum was prepped by adding 190 μl Clostridium sporogenes ATCC 3584 (˜1×10⁸ spores/ml in water) and 10 μl fetal bovine serum (FBS) to a 1.5 mL centrifuge tube and vortexing briefly to mix. The actual amount of spores and FBS added could change based on the number of samples to be tested that day, but the ratio between the two stayed constant.

Onto each piece of VITRO-SKIN® substrate 10 μl of the inoculum was placed with a pipette. The pipette tip was used to gently spread the inoculum in a circle roughly 5 mm in diameter. As a numbers control, 10 μl of the inoculum was also pipetted directly into one of the 50 mL conical tube containing 15 mL of D/E broth, this was repeated twice more. The inoculated VITRO-SKIN® substrate was left for between 30 minutes and one hour until the inoculum was visually dry.

Either before preparing the plates or during the inoculum dry time the wipes were prepared. This was done by cutting a 4″ by 6″ piece of the desired substrate. The cut substrate was weighed and solution in the amount of 3.5× the weight of the substrate was added, the amount of solution added could change depending on the saturation level of the wipe. The substrate was scrunched together to evenly distribute the solution throughout, from here on the substrate and solution combination will simply be called the wipe. The wipe was then placed into a plastic bag which was sealed to prevent it from drying out. Three wipes were prepared for each sample to be tested.

Once the inoculum had dried on the VITRO-SKIN® substrate, one piece of contaminated VITRO-SKIN® substrate was placed into one of the 50 mL conical tubes with 15 mL of D/E broth, this was repeated for a total of three pieces of VITRO-SKIN® substrates, this was the recovery control.

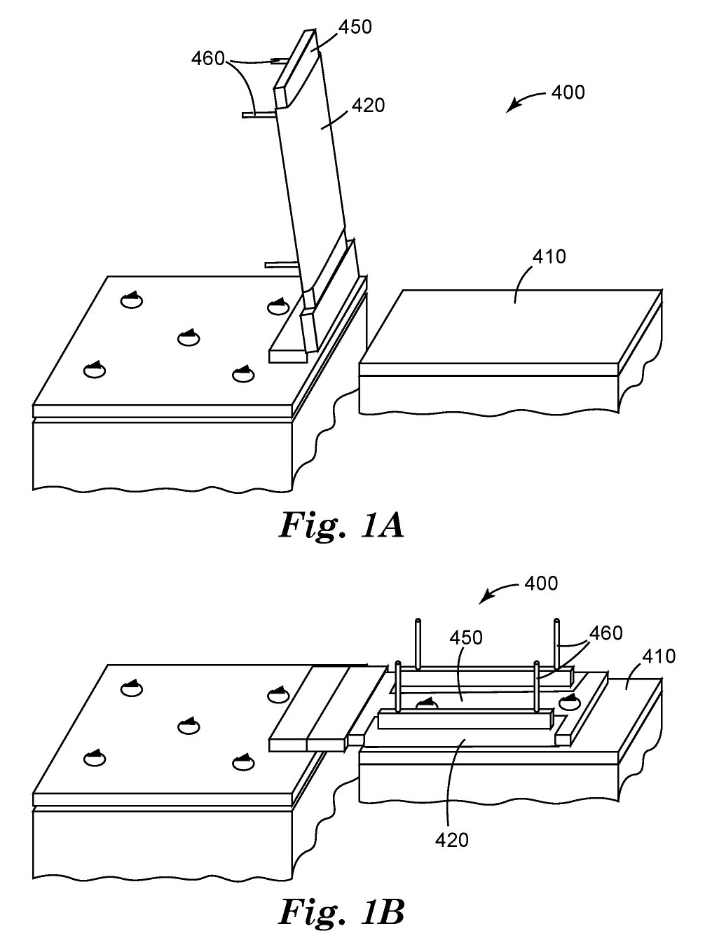

For the samples tested, a mechanical wiping device 400 was used as shown in FIGS. 1A and 1B. The wet wipe 420 was locked onto the lever arm 450 of the mechanical wiping device 400 using screw clamps 460. The lever arm 450 had a mass of about 350 g. For the two-step method, approximately 0.5 mL of the first solution was placed onto the center of the inoculated VITROSKIN® substrate attached to a stainless steel plate located on platform 410. For the one step method no solution was added to the inoculated VITROSKIN® substrate. Then the lever arm 450 with wet wipe 420 attached was lowered onto one of the contaminated plates (not shown) on platform 410 so that the inoculated VITRO-SKIN® substrate was in the center of the wipe. The mechanical wiping device 400 was switched on, with the lever arm 450 operating at a rotational speed of about 100 rpm to wipe the surface of contaminated plate for 15 seconds. After wiping the wet wipe 420 was then removed from the “wiped plate” and the VITRO-SKIN® substrate was removed and placed in a 50 mL conical tube with 15 mL of D/E. For each wipe example, this step was repeated to give n=3 wiped VITRO-SKIN® substrates.

After all the samples were collected all of the 50 mL conical tubes were sonicated for one minute then vortexed for one minute. A one milliliter aliquot was removed and added to a 1.5 mL centrifuge tube. This was done twice for each sample. The tube was then heated to 80 degrees C. for ten minutes. After cooling for three minutes 0.25 mL was removed from one tube for each sample and plated directly onto blood agar plates, for the other tube each sample was serially diluted in Butterfields Buffer from 10⁻¹ to 10⁻⁴, then 1 mL of each of the serial dilutions was plated onto 3M PETRIFILM® AC plates. Both blood agar plates and 3M PETRIFILM® AC plates were incubated in an anaerobic chamber at 37 degrees C. for approximately 20 hours. After incubation the plates were removed, counted and log reduction values were calculated.

### Working Standard

In the examples that follow, a “Working Standard” is indicated within various examples. Because the nature of the testing utilized herein, results may seem to vary from day to day, from testing personnel to testing personnel, or both. Within the different sets of examples, a single one that is the same is indicated as a Working Standard where applicable. This affords a more meaningful comparison of spore removal across examples.

### Comparative Example 1

Sontara 8005 wipes were coated (or not) with GPEI or dodecyl GPEI as discussed above. The compositions for Comparative examples 1a to 1c were prepared as discussed above. The ability of the compositions to remove spores was tested as discussed above using a one-step method on VITRO-SKIN® substrate.

| Comparative Ex. 1 | Comparative 1a | Comparative 1b | Comparative 1c |
|---|---|---|---|
| Ethanol | 70 | 70 | 70 |
| Water | 30 | 30 | 30 |
| Wipe | Sontara 8005 | Sontara 8005 | Sontara 8005 |
| Coated? | No | Yes | Yes |
| Coating Type | N/A | GPEI | Dodecyl GPEI |
| Log Reduction | 1.54 | 1.81 | 1.67 |
| Standard Deviation | 0.25 | 0.17 | 0.07 |

These comparative examples show that when a wipe contains 70% ethanol there is no statistical difference in spore removal between a coated (guanylated or dodecyl guanylated) wipe and an uncoated wipe in a one-step method. The spores likely denature on the VITRO-SKIN® substrate making it difficult to remove them from the VITRO-SKIN® substrate and to get them onto the wipe surface.

### Example 2

Sontara 8005 wipes were coated (or not) with GPEI as discussed above. The compositions for Comparative examples 2a and 2b were prepared as discussed above. The ability of the compositions to remove spores was tested as discussed above using a one-step method on VITRO-SKIN® substrate.

| Comparative Example 2 | Comparative 2a | Comparative 2b |
|---|---|---|
| Ethanol | 70 | 70 |
| Water | 30 | 30 |
| Coated? | No | Yes |
| Coating Type | N/A | GPEI |
| Wipe | Sontara 8005 | Sontara 8005 |
| Log Reduction | 1.33 | 1.30 |
| Standard Deviation | 0.20 | 0.12 |

Comparative Examples 1a and 1b were repeated on a separate day as Comparative Examples 2a and 2b and both are presented herein. The explanation relevant to the Working Standard above may be applicable in Comparative Examples as well.

### Example 3

Sontara 8005 wipes were coated with GPEI as discussed above. The compositions for Comparative Example 3a and Example 3b were prepared as discussed above. The ability of the compositions to remove spores was tested as discussed above using a two-step method on VITRO-SKIN® substrate.

| | | |
|---|---|---|
| Example 3 | Comparative 3a | 3b |

First Step - Solution added onto VITRO-SKIN ® substrate

| | | |
|---|---|---|
| Ethanol | 0 | 70 |
| Water | 100 | 30 |

Second Step - VITRO-SKIN ® substrate wiped with pre-wetted wipe

| | | |
|---|---|---|
| Tween 20 | 0.1 | 0.1 |
| Water | 99.9 | 99.9 |
| Wipe | Sontara 8005 | Sontara 8005 |
| Coated? | Yes | Yes |
| Coating Type | GPEI | GPEI |
| Log Reduction | 2.24 | 2.72 |
| Standard Deviation | 0.09 | 0.25 |

### Example 4

Sontara 8005 wipes were coated with GPEI as discussed above. The compositions for Examples 4a and 4b and Example 4c were prepared as discussed above. The ability of the compositions to remove spores was tested as discussed above using a two-step method on VITRO-SKIN® substrate.

| | | | |
|---|---|---|---|
| Example 4 | Example 4a | Example 4b | Example 4c |

First Step - Solution added onto VITRO-SKIN ® substrate

| | | | |
|---|---|---|---|
| Ethanol | 70 | 0 | 70 |
| IPA | 0 | 70 | 0 |
| ULTREZ 20 | 0 | 0 | 0.3 |
| Triethanolamine | 0 | 0 | 0.15 |
| Water | 30 | 30 | 29.55 |

Second Step - VITRO-SKIN ® substrate wiped with pre-wetted wipe

| | | | |
|---|---|---|---|
| Tween 20 | 0.1 | 0.1 | 0.1 |
| Water | 99.9 | 99.9 | 99.9 |
| Wipe | Sontara 8005 | Sontara 8005 | Sontara 8005 |
| Coated? | Yes | Yes | Yes |
| Coating Type | GPEI | GPEI | GPEI |
| Log Reduction | 2.63 | 2.47 | 1.14 |
| Standard Deviation | 0.24 | 0.29 | 0.11 |

As seen by comparing Examples 4a and 4b, no difference can be seen when changing from ethanol to IPA. However, when the solution was thickened the log reduction decreased dramatically.

### Example 5

Sontara 8005 wipes were coated with GPEI as discussed above. The compositions for Examples 5a and 5b and Comparative Example 5c were prepared as discussed above. The ability of the compositions to remove spores was tested as discussed above using a two-step method on VITRO-SKIN® substrate.

| | | | |
|---|---|---|---|
| Example 5 | Example 5a | Example 5b | Comparative 5c |

First Step - Solution added onto VITRO-SKIN ® substrate

| | | | |
|---|---|---|---|
| Tween 20 | 0 | 0.1 | 0 |
| Ethanol | 70 | 0 | 70 |
| Water | 30 | 99.9 | 30 |

Second Step - VITRO-SKIN ® substrate wiped with pre-wetted wipe

| | | | |
|---|---|---|---|
| Tween 20 | 0.1 | 0 | 0 |
| Ethanol | 0 | 70 | 70 |
| Water | 99.9 | 30 | 30 |
| Wipe | Sontara 8005 | Sontara 8005 | Sontara 8005 |
| Coated? | Yes | Yes | Yes |
| Coating Type | GPEI | GPEI | GPEI |
| Log Reduction | 2.40 | 2.24 | 0.27 |
| Standard Deviation | 0.14 | 0.16 | 0.08 |

As seen by comparing Examples 5a and 5b, there is no difference whether the 70% alcohol is on the VITRO-SKIN® substrate or water is on the VITRO-SKIN® substrate as long as the other solution is in the wipe. If alcohol is used in both places then it dramatically decreases spore removal to under a half a log, as seen in Comparative 5c.

### Example 6

| Example 6 | | | |
|---|---|---|---|
| Example | Example | Example | Example |
| 6a | 6b | 6c | 6d |

First Step - Solution added onto VITRO-SKIN ® substrate

| | | | | |
|---|---|---|---|---|
| Ethanol | 70 | 70 | 70 | 70 |
| Water | 30 | 30 | 30 | 30 |

Second Step - VITRO-SKIN ® substrate wiped with pre-wetted wipe

| | | | | |
|---|---|---|---|---|
| Tween 20 | 0.1 | 0.1 | 0.1 | 0.1 |
| Water | 99.9 | 99.9 | 99.9 | 99.9 |
| Wipe | Sontara | Sontara | Sontara | Sontara |
| | 8005 | 8005 | 8005 | 8005 |
| Coated? | Yes | Yes | Yes | Yes |
| Coating Type | GPEI | 5k PEG | Octyl GPEI | PEI |
| | | GPEI | | |
| Log Reduction | 2.98 | 2.70 | 2.99 | 3.08 |
| Standard Deviation | 0.04 | 0.17 | 0.05 | 0.09 |

As seen by comparing all of Examples 6a to 6d, all of the coatings tested provided good spore removal.

### Example 7

| Example 7 | | | |
|---|---|---|---|
| Example | Example | Example | Example |
| 7a | 7b | 7c | 7d |

First Step - Solution added onto VITRO-SKIN ® substrate

| | | | | |
|---|---|---|---|---|
| Ethanol | 70 | 70 | 70 | 70 |
| Ultrez 20 | 0 | 0.05 | 0.1 | 0.2 |
| Triethanolamine | 0 | 0.02 | 0.05 | 0.1 |
| Water | 30 | 29.93 | 29.85 | 29.7 |

Second Step - VITRO-SKIN ® substrate wipe with pre-wetted wipe

| | | | | |
|---|---|---|---|---|
| Tween 20 | 0.1 | 0.1 | 0.1 | 0.1 |
| Water | 99.9 | 99.9 | 99.9 | 99.9 |
| Wipe | Sontara | Sontara | Sontara | Sontara |
| | 8005 | 8005 | 8005 | 8005 |
| Coated? | Yes | Yes | Yes | Yes |
| Coating Type? | GPEI | GPEI | GPEI | GPEI |
| Coated? | Yes | Yes | Yes | Yes |
| Log Reduction | 2.45 | 2.61 | 2.08 | 1.75 |
| Standard Deviation | 0.12 | 0.12 | 0.28 | 0.35 |

### Example 8

| | | | |
|---|---|---|---|
| Example 8 | Example 8a | Example 8b | Comparative 8c |

First Step - Solution added onto VITRO-SKIN ® substrate

| | | | |
|---|---|---|---|
| Ethanol | 70 | 70 | 70 |
| Water | 30 | 30 | 30 |

Second Step - VITRO-SKIN ® substrate wiped with pre-wetted wipe

| | | | |
|---|---|---|---|
| Tween 20 | 0.1 | 0.1 | 0.1 |
| Water | 99.9 | 99.9 | 99.9 |
| Wipe | Sontara 8005 | Ahlstrom 200 | Sontara 8005 |
| Coated? | Yes | Yes | No |
| Coating Type | GPEI | GPEI | N/A |
| Log Reduction | 2.6 | 2.33 | 1.99 |
| Standard Deviation | 0.04 | 0.09 | 0.07 |

### Example 9

Example 9a utilized the first composition directly on the VITRO-SKIN® substrate and followed by using a GPEI-coated wipe with the second composition loaded therein. Example 9b utilized the first composition loaded in a non-coated wipe and followed by using a non-coated wipe with the second composition loaded therein. Example 9c utilized the first composition loaded in a non-coated wipe and followed by using a GPEI-coated wipe with the second composition loaded therein. Example 9d utilized the first composition loaded in a coated wipe and followed by using a coated wipe with the second composition loaded therein.

| Example 9 | | | |
|---|---|---|---|
| Example | Example | Example | Example |
| 9a | 9b | 9c | 9d |

First Step - Solution dispense in onto VITRO-SKIN ® or onto wipe

| | Solution | Solution dispensed in wipe and | | |
|---|---|---|---|---|
| | dispensed | wiped VITRO-SKIN ® | | |
| | onto VITRO- | substrate | | |
| | SKIN ® | | | |
| Ethanol | 70 | 70 | 70 | 70 |
| Water | 30 | 30 | 30 | 30 |
| Wipe | N/A | Sontara | Sontara | Sontara |
| | | 8005 | 8005 | 8005 |
| Wipe 1 Coated? | N/A | No | No | Yes |
| Coating Type | N/A | N/A | N/A | GPEI |

Second Step - VITRO-SKIN ® substrate wiped with pre-wetted wipe

| | | | | |
|---|---|---|---|---|
| Tween 20 | 0.1 | 0.1 | 0.1 | 0.1 |
| Water | 99.9 | 99.9 | 99.9 | 99.9 |
| Wipe 2 Coated? | Yes | No | Yes | Yes |
| Coating Type | GPEI | N/A | GPEI | GPEI |
| Wipe | Sontara | Sontara | Sontara | Sontara |
| | 8005 | 8005 | 8005 | 8005 |
| Log Reduction | 2.91 | 2.82 | 3.08 | 3.37 |
| Standard Deviation | 0.05 | 0.10 | 0.08 | 0.14 |

Thus, embodiments of methods of removing spores are disclosed. The implementations described above and other implementations are within the scope of the following claims. One skilled in the art will appreciate that the present disclosure can be practiced with embodiments other than those disclosed. The disclosed embodiments are presented for purposes of illustration and not limitation.

### EXEMPLARY EMBODIMENTS

1. A method comprising:

contacting a skin surface with a first liquid composition; and

then contacting the skin surface with a cationic coated article loaded with a second liquid composition, while at least some portion of the first liquid composition remains on the skin surface, wherein at least one and only one of the first or the second composition comprises greater than or equal to 60 wt % of at least one alcohol.

2. The method according to embodiment 1, wherein the step of contacting a first composition with the skin surface is accomplished via contacting the skin surface with only the first composition.

3. The method according to any of embodiments 1 or 2, wherein the step of contacting the surface with the first composition comprises applying the composition to the skin surface.

4. The method according to embodiment 3, wherein the step of applying the composition to the skin surface comprises spraying, dispensing, dipping, pouring, or some combination thereof on to the skin surface.

5. The method according to any of embodiments 1 to 4 further comprising subjecting the skin surface in contact with the first composition to mechanical action before contacting the skin surface to the cationic coated article loaded with a second composition.

6. The method according to embodiment 5, wherein the mechanical action lasts at least about 5 seconds.

7. The method according to embodiment 5, wherein the step of subjecting the skin surface in contact with the first composition to mechanical action comprises rubbing the skin surface, moving an article across the skin surface, or some combination thereof.

8. The method according to any of embodiments 1 to 7, wherein the first composition comprises greater than or equal to 60 wt % of at least one alcohol.

9. The method according to embodiment 8, wherein there is at least about 65 wt % of at least one alcohol in the first composition based on the total weight of the first composition.

10. The method according to embodiment 8, wherein there is at least about 70 wt % of at least one alcohol in the first composition based on the total weight of the first composition.

11. The method according to embodiment 8, wherein there is not greater than 95 wt % of at least one alcohol in the first composition based on the total weight of the first composition.

12. The method according to any of embodiments 1 to 7, wherein the second composition comprises greater than or equal to 60 wt % of at least one alcohol.

13. The method according to embodiment 12, wherein there is at least about 65 wt % of at least one alcohol in the second composition based on the total weight of the second composition.

14. The method according to embodiment 12, wherein there is at least about 70 wt % of at least one alcohol in the second composition based on the total weight of the second composition.

15. The method according to embodiment 12, wherein there is not greater than 95 wt % of at least one alcohol in the second composition based on the total weight of the second composition.

16. The method according to any of embodiments 1 to 15, wherein the cationic coated article comprises a cationic coated wipe.

17. The method according to any of embodiments 1 to 16, wherein the cationic coated article comprises a guanidinyl-containing polymer.

18. The method according to embodiment 17, wherein the guanidinyl-containing polymer is a reaction product of (a) a guanylating agent and (b) a carbonyl-containing polymer precursor or an amino-containing polymer precursor.

19. The method according to embodiment 17, wherein the guanidinyl-containing polymer is of the Formula (I):

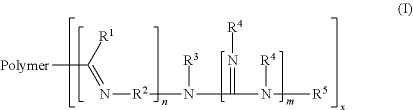

wherein:

R¹ is hydrogen, C₁-C₁₂(hetero)alkyl, a C₅-C₁₂ (hetero)aryl, or a residue of the polymer chain;

R² is a covalent bond, a C₂ to C₁₂ (hetero)alkylene, or a C₅-C₁₂ (hetero)arylene;

R³ is hydrogen, C₁-C₁₂ (hetero)alkyl, C₅-C₁₂ (hetero)aryl, or a residue of the polymer chain when n is 0;

each R⁴ is independently hydrogen, C₁-C₁₂ (hetero)alkyl, C₅-C₁₂ (hetero)aryl;

R⁵ is hydrogen, C₁-C₁₂ (hetero)alkyl, or C₅-C₁₂ (hetero)aryl, or —N(R⁴)₂;
n is 0 or 1;
m is 1 or 2; and
x is an integer equal to at least 1.

20. The method according to embodiment 19, wherein the guanidinyl-containing polymer is of Formula (II):

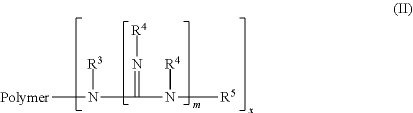

21. The method according to embodiment 19, wherein the guanidinyl-containing polymer is of Formula (IV):

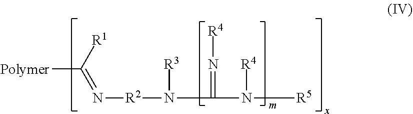

22. The method according to any one of embodiments 17 to 19 or 21, wherein the guanidinyl-containing polymer is a reaction product of (a) a guanylating agent and (b) a carbonyl-containing polymer precursor, and wherein 1 to 90 mole percent of the carbonyl groups of the carbonyl-containing polymer precursor are reacted with the guanylating agent.

23. The method according to any one of embodiments 17 to 19 or 21, wherein the guanidinyl-containing polymer is a reaction product of (a) a guanylating agent and (b) a carbonyl-containing polymer precursor, and wherein the guanidinyl-containing polymer is crosslinked with a N,N′-(hetero)alkylenebis(meth)acrylamide.

24. The method according to any one of embodiments 17 to 20, wherein the guanidinyl containing polymer is a reaction product of a (a) guanylating agent and (b) an amino-containing polymer precursor, and wherein 1 to 90 mole percent of the amino groups of the amino-containing polymer precursor are reacted with the guanylating agent.

25. The method according to any one of embodiments 17 to 20, wherein the guanidinyl-containing polymer is a reaction product of (a) a guanylating agent and (b) an amino-containing polymer, and wherein the guanidinyl-containing polymer is crosslinked with a polyglycidylether.

26. The wipe according to any one of embodiments 17 to 25, wherein guanidinyl-containing polymer is present in an amount of 0.1 weight percent to 10 weight percent based on a total weight of the wipe.

27. The method according to any of embodiments 1 to 26, wherein the at least one alcohol is selected from ethanol, n-propanol, 2-propanol or mixtures thereof.

28. The method according to any of embodiments 1 to 27, wherein the first composition, the second composition or both further comprises an antimicrobial agent in addition to the at least one alcohol.

29. The method according to embodiment 28, wherein the antimicrobial agent is cationic.

30. The method according to any of embodiments 1 to 29, wherein the first composition, the second composition or both further comprise not greater than 0.3 wt % thickener based on the total weight of the first composition, the second composition, or both respectively.

31. The method according to any of embodiments 1 to 30, wherein the first composition comprises not greater than 0.2 wt % thickener based on the total weight of the first composition.

32. The method according to any of embodiments 1 to 30, wherein the first composition comprises not greater than 0.1 wt % thickener based on the total weight of the first composition.

33. The method according to any of embodiments 1 to 30, wherein the first composition comprises not greater than 0.05 wt % thickener based on the total weight of the first composition.

34. The method according to any of embodiments 1 to 33, wherein the step of contacting a first composition with the skin surface is accomplished via contacting the skin surface with the first composition in contact with a second article.

35. The method according to embodiment 34, wherein the second article is a wipe.

36. The method according to embodiment 35, wherein the article is a cationic coated wipe.

1-15. (canceled)

16. A method comprising:
contacting a surface with a first liquid composition; and
then contacting the surface with a cationic coated article loaded with a second liquid composition, while at least some portion of the first liquid composition remains on the surface, wherein at least one and only one of the first or the second composition comprises greater than or equal to 60 wt % of at least one alcohol.

17. The method according to claim 16, wherein the surface is skin.

18. The method according to claim 16, wherein the step of contacting the surface with the first composition comprises applying the composition to the skin surface.

19. The method according to claim 18, wherein the step of applying the composition to the skin surface comprises spraying, dispensing, dipping, pouring, or some combination thereof on to the skin surface.

20. The method according to claim 16 further comprising subjecting the skin surface in contact with the first composition to mechanical action before contacting the skin surface to the cationic coated article loaded with a second composition.

21. The method according to claim 16, wherein the first composition comprises greater than or equal to 60 wt % of at least one alcohol.

22. The method according to claim 16, wherein the second composition comprises greater than or equal to 60 wt % of at least one alcohol.

23. The method according to claim 16, wherein the cationic coated article comprises a cationic coated wipe.

24. The method according to claim 16, wherein the cationic coated article comprises a guanidinyl-containing polymer.

25. The method according to claim 24, wherein the guanidinyl-containing polymer is a reaction product of (a) a guanylating agent and (b) a carbonyl-containing polymer precursor or an amino-containing polymer precursor.

26. The method according to claim 24, wherein the guanidinyl-containing polymer is of the Formula (I): 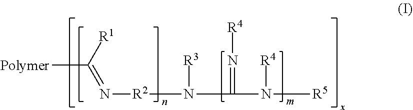
wherein:
R¹ is hydrogen, C₁-C₁₂(hetero)alkyl, a C₅-C₁₂ (hetero)aryl, or a residue of the polymer chain;
R² is a covalent bond, a C₂ to C₁₂ (hetero)alkylene, or a C₅-C₁₂ (hetero)arylene;
R³ is hydrogen, C₁-C₁₂ (hetero)alkyl, C₅-C₁₂ (hetero)aryl, or a residue of the polymer chain when n is 0;
each R⁴ is independently hydrogen, C₁-C₁₂ (hetero)alkyl, C₅-C₁₂ (hetero)aryl;
R⁵ is hydrogen, C₁-C₁₂ (hetero)alkyl, or C₅-C₁₂ (hetero)aryl, or —N(R⁴)₂;
n is 0 or 1;
m is 1 or 2; and
x is an integer equal to at least 1.

27. The method according to claim 24, wherein the guanidinyl-containing polymer is of Formula (II):

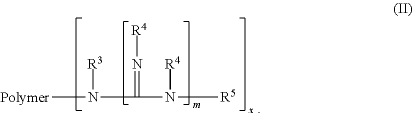

28. The method according to claim 24, wherein the guanidinyl-containing polymer is of Formula (IV):

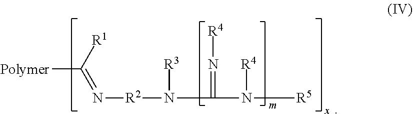

29. The method according to claim 16, wherein the at least one alcohol is selected from ethanol, n-propanol, 2-propanol or mixtures thereof.

30. The method according to claim 16, wherein the step of contacting a first composition with the surface is accomplished via contacting the surface with the first composition in contact with a wipe.

31. A method comprising:
contacting a surface with a first liquid composition; and
then contacting the surface with a cationic coated article loaded with a second liquid composition, while at least some portion of the first liquid composition remains on the surface, wherein only the first composition comprises greater than or equal to 60 wt % of at least one alcohol.

32. A method comprising:
contacting a skin surface with a first liquid composition; and
then contacting the skin surface with a cationic coated article loaded with a second liquid composition, while at least some portion of the first liquid composition remains on the skin surface, wherein only the second composition comprises greater than or equal to 60 wt % of at least one alcohol.
